(19) United States
(12) Patent Application Publication
(10) Pub. No.: US 2025/0380980 A1
(43) Pub. Date: Dec. 18, 2025

(54) SYSTEMS AND METHODS FOR UTERINE FIBROID ABLATION

(71) Applicant: Gynesonics, Inc., Redwood City, CA (US)

(72) Inventors: Harry KWAN, San Francisco, CA (US); Jiayu CHEN, Palo Alto, CA (US); Oren MOSHER, Castro Valley, CA (US)

(21) Appl. No.: 18/879,178

(22) PCT Filed: Jun. 27, 2023
(86) PCT No.: PCT/US2023/069208

§ 371 Date: Dec. 26, 2024

Related U.S. Application Data
(60) Provisional application No. 63/356,223, filed on Jun. 28, 2022.

(51) Int. Cl. A61B 18/14; A61B 17/34; A61B 18/00; A61B 18/12; A61B 34/20; A61B 90/00

(52) U.S. Cl. CPC A61B 18/1477; A61B 18/1206; A61B 34/20; A61B 2017/3413; A61B 2018/0016; A61B 2018/00559; A61B 2018/00577; A61B 2018/00607; A61B 2018/00642; A61B 2018/00672; A61B 2018/00678; A61B 2018/00702; A61B 2018/00714; A61B 2018/00767; A61B 2018/00791; A61B 2018/00898; A61B 2018/1475; A61B 2034/2063; A61B 2090/3782

(57) ABSTRACT

Various systems, devices, and methods for tissue penetration are disclosed. The system includes an ablation element that penetrates the tissue and a radiofrequency generator that delivers energy to the ablation element. The radiofrequency generator may have two modes: a cutting or insertion mode and an ablation or coagulation mode. The cutting or insertion mode is used to cut or penetrate the tissue by providing a voltage or power modulation that acts to cut or soften tissue contacted by the ablation element. The ablation or coagulation mode is used to ablate or coagulate the tissue.

### CROSS-REFERENCE TO RELATED APPLICATION AND INCORPORATION BY REFERENCE

This application claims the benefit of U.S. Provisional Application No. 63/356,223, filed Jun. 28, 2022, the entirety of which is hereby incorporated by reference.

All publications, patents, and patent applications mentioned in this specification are herein incorporated by reference to the same extent as if each individual publication, patent, or patent application was specifically and individually indicated to be incorporated by reference.

### BACKGROUND OF THE INVENTION

### Technical Field

The present disclosure relates to medical systems, devices, and methods, particularly for uterine fibroid ablation. The present disclosure also relates to inserting instruments into target tissue.

### Description of the Related Art

Current medical treatments of organs and tissues within a patient's body often use a needle or other elongate body for delivery of energy, therapeutic agents or the like. Optionally the methods use ultrasound imaging to observe and identify a treatment or diagnostic target and track the position of the needle relative to the target.

Treatment for uterine fibroids has been proposed which relies on the transvaginal or laparoscopic positioning of a treatment device in the patient's uterus. A radiofrequency or other energy or therapeutic delivery needle is deployed from the device into the fibroid, and energy and/or therapeutic substances are delivered in order to ablate or treat the fibroid. To facilitate locating the fibroids and positioning the needles within the fibroids, the device includes an ultrasonic imaging array with an adjustable field of view in a generally forward or lateral direction relative to an axial shaft which carries the needle. The needle is advanced from the shaft and across the field of view so that the needle can be visualized and directed into the tissue and the targeted fibroid.

Current systems, devices, and methods for therapeutic or diagnostic procedures, such as for treatment for uterine fibroids, may be less than ideal in at least some respects. For example, many current devices for tissue ablation require the insertion of an ablation element into the target tissue. However, target tissue provides resistance against many current devices configured to penetrate into the target tissue. For example, when a user tries to insert an instrument into target tissue-especially dense target tissue, such as a fibroid-the force required to penetrate the target tissue can cause the target tissue to substantially deform or otherwise resist the insertion of the instrument.

In light of the above, improved systems, devices, and methods for penetrating into dense target tissue, such as a fibroid are desired. Such systems, devices, and methods would address at least some of the drawbacks above and would, for example, be easier to be used for a greater variety of therapeutic and diagnostic procedures.

### SUMMARY

The embodiments disclosed herein each have several aspects no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices and methods.

The present disclosure relates to medical systems, devices, and methods, particularly for but not limited to uterine fibroid ablation. Embodiments of the present disclosure provide ablation elements configured to penetrate target tissue, and provide a radiofrequency generator configured to delivery energy to the ablation elements. Furthermore, the radiofrequency generator of embodiments of the present disclosure can comprise a cutting or insertion mode configured to cut through target tissue. The cutting or insertion mode can provide for a voltage oscillation configured to cut tissue contacted by the ablation elements while the ablation elements are being inserted into the target tissue, before the ablation procedure begins. The use of such a cutting or insertion mode during the insertion of ablation elements into target tissue can reduce the force required to insert the ablation elements into the target tissue, which in turn can make the insertion easier and more accurate.

In some aspects, a system for penetrating target tissue is provided. The system may comprise an ablation instrument, and a controller configured to control the delivery of energy to the ablation instrument, wherein the controller is configured to monitor a temperature measured by the ablation instrument at an interface with the target tissue and maintain the temperature between about 80° C. and about 115° C. as the ablation instrument penetrates into the target tissue.

The system described above or as provided in other aspects described herein may comprise one or more of the following features. The ablation instrument may comprise an introducer and a plurality of electrode ablation needles extendible from a retracted position within the introducer. The temperature measured by the ablation instrument is measured by a thermocouple positioned on one of the electrode ablation needles when the plurality of electrode ablation needles is retracted within the introducer. The plurality of electrode ablation needles may comprise a central electrode extendible from a retracted position within the introducer, and wherein the thermocouple is positioned on the central electrode. The controller may be configured to modulate power delivered to the ablation instrument to maintain the temperature between about 80° C. and about 115° C. as the ablation instrument penetrates into the target tissue. The controller may be configured to modulate power delivered to the ablation instrument as the ablation instrument penetrates into the target tissue to provide an oscillation in the temperature at the interface with the target tissue. The controller may be configured to provide an alert when the temperature at the interface is between about 80° C. and about 115° C. to provide an indication to begin penetration by the ablation instrument into the target tissue. The system may further comprise an ultrasonic imaging device configured to provide for visualization of the ablation instrument as the ablation instrument penetrates the target tissue. The ultrasonic imaging device may be coupled to the ablation instrument. The controller may be configured to control the delivery of energy to the ablation instrument to ablate the target tissue after the ablation instrument has penetrated into the target tissue. The controller may be configured to control the delivery of energy to the ablation instrument to maintain a substantially constant temperature at the target tissue to ablate the target tissue. The system may further comprise a radiofrequency generator configured to deliver energy to the ablation instrument while the ablation instrument penetrates into the target tissue.

In some aspects, a method of penetrating target tissue is provided. The method may comprise delivering an ablation instrument into contact with a target tissue, delivering energy to the ablation instrument to cause a temperature measured by the ablation instrument at an interface with the target tissue to rise to between about 80° C. and about 115° C., and advancing the ablation instrument to penetrate into the target tissue while modulating the power to maintain the temperature at the interface with the target tissue between about 80° C. and about 115° C.

The method described above or as described in other aspects described herein may comprise one or more of the following features. The ablation instrument may penetrate into the target tissue while the temperature maintained at the interface with the target tissue oscillates. The method may further comprise ablating the target tissue by delivering energy to the ablation instrument to maintain the temperature at the interface with the target tissue at a substantially constant temperature. The ablation instrument may comprise an introducer and a plurality of electrode ablation needles extendible from a retracted position within the introducer, wherein the plurality of electrode ablation needles is at least partially extended from the introducer while maintaining the temperature at the interface with the target tissue between about 80° C. and about 115° C.

In some aspects, a system for penetrating target tissue. The system may comprise an ablation element configured to penetrate the target tissue, a radiofrequency generator configured to deliver energy to the ablation element, wherein the radiofrequency generator comprises a cutting or insertion mode configured to cut through target tissue, wherein the cutting or insertion mode provides for a power oscillation configured to cut tissue contacted by the ablation element, and an ablation or coagulation mode configured to ablate and/or coagulate the target tissue, wherein the ablation or coagulation mode provides for an increase in power and then a decrease in power after the target tissue reaches a target temperature.

The system described above or as provided in other aspects described herein may comprise one or more of the following features. The cutting or insertion mode may be configured to maintain a substantially constant surface temperature of the ablation element. The cutting or insertion mode may be configured to cause a rapid increase in temperature in tissue in contact with the ablation element. The radiofrequency generator may be configured to be controlled to provide a limit on temperature during the cutting or insertion mode and during the ablation or coagulation mode, wherein the limit on temperature during the cutting or insertion mode is lower than the limit on temperature during the ablation or coagulation mode. The system may be further configured to provide a mechanical vibration or cutting force during the cutting or insertion mode. The ablation element may comprise an introducer. The ablation element may comprise a plurality of electrode ablation needles. The system may further comprise a controller configured to control the delivery of energy to the ablation element, wherein the controller is configured to monitor a temperature measured by the ablation element at an interface with the target tissue and maintain the temperature between about 80° C. and about 115° C. as the ablation element penetrates into the target tissue. The target tissue may be a uterine fibroid.

In some aspects, a method of uterine fibroid ablation is provided. The method may comprise delivering an ablation element into contact with a uterine fibroid, delivering radiofrequency energy according to a cutting or insertion mode to the ablation element while the ablation element is in contact with the uterine fibroid, wherein in the cutting or insertion mode a voltage or power is modulated based on a temperature measured at an interface between the ablation element and the uterine fibroid to assist the ablation element in penetrating into the uterine fibroid, and after the ablation element penetrates into the uterine fibroid, delivering radiofrequency energy according to a coagulation mode, wherein in the coagulation mode a voltage or power applied to the ablation element is sufficient to ablate the uterine fibroid.

The method described above or as described in other aspects described herein may comprise one or more of the following features. The voltage or power may be oscillated in the cutting or insertion mode. The voltage or power may be modulated in the cutting or insertion mode to maintain a temperature at the interface between about 80° C. and about 115° C. The voltage or power in the coagulation mode may increase and then decrease after tissue in contact with the ablation element reaches a target temperature. Delivering radiofrequency energy according to the cutting or insertion mode may preheat the uterine fibroid. Delivering radiofrequency energy according to the cutting or insertion mode may soften the uterine fibroid thereby allowing the ablation element to penetrate the uterine fibroid without substantially deforming the uterine fibroid upon penetration. Delivering radiofrequency energy according to the cutting or insertion mode may maintain a substantially constant surface temperature of the ablation element. The substantially constant surface temperature of the ablation element may be between about 80° C. and about 115° C. Delivering radiofrequency energy according to the cutting or insertion mode may be controlled to a maximum output not to exceed 70 watts. Delivering radiofrequency energy according to the cutting or insertion mode may be controlled not to exceed 30 seconds.

### BRIEF DESCRIPTION OF THE DRAWINGS

Certain features of this disclosure are described below with reference to the drawings. The illustrated implementations are intended to illustrate, but not to limit, the implementations. Various features of the different disclosed implementations can be combined to form further implementations, which are part of this disclosure.

FIG. 1A shows a perspective view of an imaging component, in accordance with some embodiments.

FIG. 1B shows a side, cross-sectional view of the imaging component of FIG. 1A, in accordance with some embodiments.

FIG. 1C shows a side, cross-section view of an imaging component having a shaft with a circular cross-section, in accordance with some embodiments.

FIG. 1D shows a side, cross-sectional view of an imaging component with edges bent inward towards the interior of the cavity, in accordance with some embodiments.

FIG. 1E shows a magnified, perspective view of a distal end of the imaging component of FIG. 1A comprising a cavity, in accordance with some embodiments.

FIG. 2 shows a magnified, perspective view of a distal end of the imaging component of FIG. 1A with a radiofrequency ablation instrument disposed within the shaft of the imaging component, in accordance with some embodiments.

FIG. 3A shows an assembly view of an imaging system comprising the imaging component of FIG. 1A and an optical scope instrument, in accordance with some embodiments.

FIG. 3B shows an assembly view of the imaging system of FIG. 3A illustrating an attachment mechanism of the system, in accordance with some embodiments.

FIG. 4 shows a magnified, perspective view of a shaft of the imaging component of FIG. 1A wherein the shaft of the imaging component is flexible, in accordance with some embodiments.

FIG. 5A illustrates a perspective view of a system for diagnosing and/or providing therapy, including an imaging component configured to be removably coupled to multiple therapeutic and/or diagnostic instruments, in accordance with some embodiments. FIG. 5A shows the imaging component and the therapeutic and/or diagnostic instrument being separated.

FIG. 5B illustrates a perspective view of the system of FIG. 5A, with the therapeutic and/or diagnostic instrument being in a ready position to be removably coupled to the imaging component, in accordance with some embodiments.

FIG. 5C illustrates a perspective view of the system of FIG. 5A, with the therapeutic and/or diagnostic instrument being removably coupled to the imaging component, in accordance with some embodiments.

FIG. 6 shows a schematic illustration of the system of the present invention comprising a system controller, an image display, and a treatment probe having a deployable needle assembly and imaging transducer.

FIG. 7A shows a schematic of the imaging component of FIG. 1A positioned within a uterus to image tissue thereof, in accordance with some embodiments.

FIG. 7B shows a surgical field image captured as in FIG. 7A that would be visible on a display, showing safety and treatment boundaries, in accordance with some embodiments.

FIG. 7C shows a surgical field image combining both a virtual image showing safety and treatment boundaries and the physical presence of an introducer, in accordance with some embodiments.

FIG. 7D shows a surgical field image combining both a virtual image showing safety and treatment boundaries as well as the physical presence of an introducer and electrode ablation needles or tines, in accordance with some embodiments.

FIG. 8 shows a graph of power over time of each of a cutting or insertion mode and an ablation mode in treating tissue, in accordance with some embodiments.

FIG. 9 shows a graph that displays power, temperature, and depth of an introducer and electrodes over time during a cutting or insertion mode, in accordance with some embodiments.

FIG. 10. shows the graph of FIG. 9 of each of a cutting or insertion mode and an ablation mode in treating tissue, in accordance with some embodiments.

FIG. 11 shows a schematic side-sectional view of the distal tip of an introducer, displaying the location of a central electrode ablation needle in its retracted position, in accordance with some embodiments.

FIG. 12 shows a flow chart depicting a method of cutting and ablating tissue, in accordance with some embodiments.

### DETAILED DESCRIPTION OF THE PREFERRED EMBODIMENTS

Various features and advantages of this disclosure will now be described with reference to the accompanying figures. The following description is merely illustrative in nature and is in no way intended to limit the disclosure, its application, or uses. This disclosure extends beyond the specifically disclosed implementations and/or uses and obvious modifications and equivalents thereof. Thus, it is intended that the scope of this disclosure should not be limited by any particular implementations described below. The features of the illustrated implementations can be modified, combined, removed, and/or substituted as will be apparent to those of ordinary skill in the art upon consideration of the principles disclosed herein. Furthermore, implementations disclosed herein can include several novel features, no single one of which is solely responsible for its desirable attributes or which is essential to practicing the systems, devices, and/or methods disclosed herein.

Certain embodiments of the present disclosure are directed to therapeutic devices, and associated methods and systems, that incorporate a cutting or insertion mode to facilitate the insertion of ablation elements into target tissue. Examples of these devices, methods and systems are described in the examples below, followed by examples of how these devices, methods and systems may be applied to uterine fibroid ablation. However, the improvements described herein are not limited to uterine fibroid ablation, and may be incorporated into any of the diagnostic and/or therapeutic devices, which may also incorporate imaging components, described herein.

Examples of Diagnostic and/or Therapeutic Devices with Imaging Devices

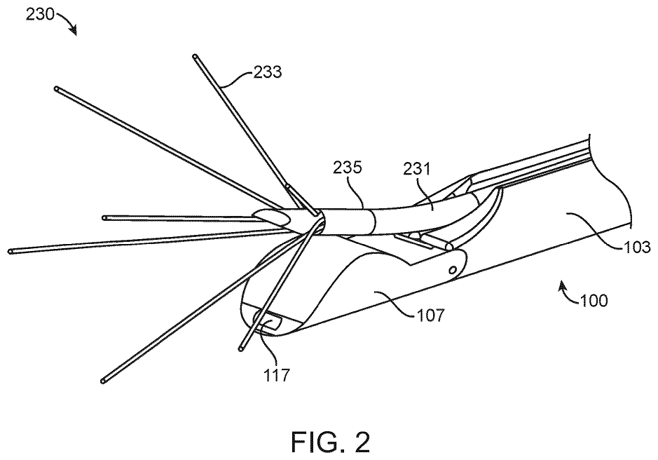

Embodiments of the present disclosure provide systems, devices, and methods for providing therapeutic and diagnostic access to tissue, while the tissue is being imaged by an imaging component. The imaging component can comprise a cavity extending across (e.g., along) the length of a shaft, wherein the cavity may be configured to removably receive at least one of a plurality of different instruments (e.g., the ablation instrument 230 of FIG. 2). In some embodiments, the cavity of the imaging component may be partially open to an exterior of the shaft. The imaging component may comprise an imaging transducer at the distal end of the shaft. Additionally, the shaft of the imaging component may be configured such that additional therapeutic and/or diagnostic instruments/attachments may be removed and/or received and/or inserted during a medical procedure without disturbing the imaging component. Additionally or alternatively, the imaging component may remain in situ while the therapeutic and/or diagnostic instrument is received and/or removed. In some embodiments, the imaging component may be used without an additional therapeutic and/or diagnostic instrument coupled thereto. In some embodiments, the imaging component may be inserted and/or removed from a patient lumen without the presence of a therapeutic and/or diagnostic instrument. Such an imaging component may be used during a medical procedure such as, for example, non-invasive, minimally invasive, and/or laparoscopic surgery.

Embodiments of the present disclosure may improve upon existing methods for imaging and treating a lesion in a tissue tract for procedures where multiple instruments may be required to diagnose and/or provide therapy during a single procedure. For example, an imaging component may be used for diagnosis; then a biopsy attachment may be inserted for a pathology sample; then an ablation attachment may be inserted for ablating any lesions; and then a further attachment or instrument may be inserted to perform additional procedures such a deliver drugs, implants, and/or therapeutic and/or diagnostic agents. The imaging component of the present disclosure may facilitate the insertion and removal of medical instruments by providing a shaft with atraumatic edges and a cavity configured to receive a plurality of different instruments. Additionally or alternatively, the imaging component may be used independently of an additional instrument or attachment. In such embodiments, the edges of the cavity may be smooth or rounded such that the edges may not catch on the patient tissue when used alone.

The cavity of an imaging component may improve upon existing methods for imaging and treatment by providing a cavity of an imaging component which may be easier to clean than a component with a closed cavity or lumen. The cavity of an imaging component may improve on existing methods for imaging and treatment by facilitating manufacture of the imaging component. Embodiments of the present disclosure may lower treatment cost by providing an imaging component with a disposable tube. Embodiments of the present disclosure may lower treatment costs by providing a reusable imaging component with a cavity into which disposable instruments (e.g., the ablation instrument 230 of FIG. 2) may be inserted. Embodiments of the imaging component may provide a shaft which aligns the instrument with the ultrasound image at all times. Embodiments of the present disclosure may accommodate various instruments with different sizes and shapes. Embodiments of the present disclosure may provide a scale or position information to assist insertion of an instrument.

The systems and methods of the present disclosure may be particularly useful in the treatment of fibroids in a patient uterus. The imaging component may be deployed transvaginally and transcervically into the uterus, or in other cases, laparoscopically into and through an exterior of the uterus or other organ or tissue tract. The imaging component may be used in conjunction with an additional instrument such as therapy electrodes; diagnostic electrodes and/or needles; a tissue ablation element (e.g., the ablation instrument 230 of FIG. 2), such as for example a radiofrequency ablation element, an ultrasonic ablation element, a heat-based ablation element, a cryoablation element, etc.; and/or other instrument suitable to be disposed within the cavity of the imaging component. Additionally or alternatively, the additional instrument may be used to deliver drugs, implants, or other therapeutic agents to the tissue to be treated. Additionally or alternatively, the tissue ablation element may comprise embodiments or variations of the needle/tine assemblies of commonly assigned U.S. Pat. Nos. 8,206,300, 8,262,574, and 8,992,427, the contents of which are incorporated herein by references.

Embodiments of the present disclosure may improve upon at least some of the systems and methods of the commonly assigned references by providing a shaft of an imaging component with atraumatic edges to enable use of the imaging component alone. In some embodiments, embodiments of the present disclosure may improve upon the ability to remove and/or receive an additional instrument (e.g., the ablation instrument 230 of FIG. 2) by providing an imaging system without an attachment mechanism located in at least the portion of the system to be positioned in situ. In such an embodiment, the imaging component shaft may be non-cylindrically symmetric (e.g., oval or rectangular in cross-section) in order to reference the rotation of the additional instrument relative to the imaging component shaft. In some embodiments, the present disclosure may additionally or alternatively provide a shaft of an imaging component with a small angled portion to minimize damage risk to a surface of an imaging transducer surface by an instrument. Additionally or alternatively, the imaging component may comprise a disposable tube inserted within the cavity to provide, among many possible purposes, a working channel for inserting additional instruments with different diameters and making the system easier to clean.

The imaging components described herein may be used in a surgical procedure to provide a real time image of a target structure to be treated, including projecting safety and treatment boundaries as described in commonly assigned U.S. Pat. Nos. 8,088,072 and 8,262,577, the contents of which are incorporated by reference. The imaging components described herein may be useful for both imaging and treating uterine fibroids as described in commonly assigned U.S. Pat. No. 7,918,795, which is incorporated herein by reference. Other commonly assigned patents and published applications describing probes useful for treating uterine fibroids which may be used with the imaging components described herein include U.S. Pat. Nos. 7,815,571, 7,874,986, 8,506,485, 9,357,977, and 9,517,047, which are incorporated herein by reference. Additional, commonly assigned patent applications describing systems for establishing and adjusting displayed safety and treatment zone boundaries which may be used in conjunction with the imaging components described herein include: U.S. Pat. Pub. No. 2014/0073910 (now U.S. Pat. No. 9,861,336); U.S. Pat. Pub. No. 2019/0350648; U.S. Pat. No. 8,992,427; U.S. Pat Pub. No. 2018/0132927 (now U.S. Pat. No. 11,219,483); and P.C.T. Pub. No. WO2018/089523, which are each incorporated herein by reference. Commonly assigned P.C.T. Pub. No. WO2018/089523, further describes mapping and planning system which may be used in conjunction with the imaging components described herein, is also incorporated herein by reference.

In some embodiments, the systems and methods of the present disclosure may provide an imaging component to be used in a variety of diagnostic and therapeutic procedures. Some embodiments may provide methods and systems to perform therapy or diagnosis on a volume of tissue. A volume of tissue may comprise a patient organ. A patient organ or bodily cavity may comprise for example: muscles, tendons, a mouth, a tongue, a pharynx, an esophagus, a stomach, an intestine, an anus, a liver, a gallbladder, a pancreas, a nose, a larynx, a trachea, lungs, a kidneys, a bladder, a urethra, a uterus, a vagina, an ovary, testes, a prostate, a heart, an artery, a vein, a spleen, a gland, a brain, a spinal cord, a nerve, etc. Some embodiments provide systems and methods suitable for laparoscopic surgery. Some embodiments provide systems and methods suitable for non-invasive surgery. Some embodiments provide systems and methods suitable for minimally invasive surgery. Some embodiments provide systems and methods suitable for robotic or robot assisted surgery.

Reference will now be made in detail to various embodiments, examples of which are illustrated in the accompanying drawings. In the following detailed description, numerous specific details are set forth in order to provide a thorough understanding of the invention and the described embodiments. However, the invention is optionally practiced without these specific details. In other instances, well-known methods, procedures, components, and circuits have not been described in detail so as not to unnecessarily obscure aspects of the embodiments.

It will be understood that, although the terms “first,” “second,” etc. are optionally used herein to describe various elements, these elements should not be limited by these terms. These terms are only used to distinguish one element from another. For example, a first instrument could be termed an instrument sensor, and, similarly, a second instrument could be termed a first instrument, without changing the meaning of the description, so long as all occurrences of the “first instrument” are renamed consistently and all occurrences of the second instrument are renamed consistently. The first instrument and the second instrument are both instruments, but they are not the same instrument.

The terminology used herein is for the purpose of describing particular embodiments only and is not intended to be limiting of the claims. As used in the description of the embodiments and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” is optionally construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” is optionally construed to mean “upon determining” or “in response to determining” or “in accordance with a determination” or “upon detecting” or “in response to detecting” that the stated condition precedent is true, depending on the context.

For ease of explanation, the figures and corresponding description below may be described below with reference to uterine imaging, specifically, in conjunction with the diagnosis and ablation and/or treatment of uterine fibroids. However, one of skill in the art will recognize that a similar imaging component may be used with similar instruments in other therapeutic applications for example: instruments for tissue biopsy, for drug delivery, for fluid infusion and/or aspiration, and for the treatment of cancers, tumors, fibroids, and other masses, malignant or benign, in any suitable bodily lumen.

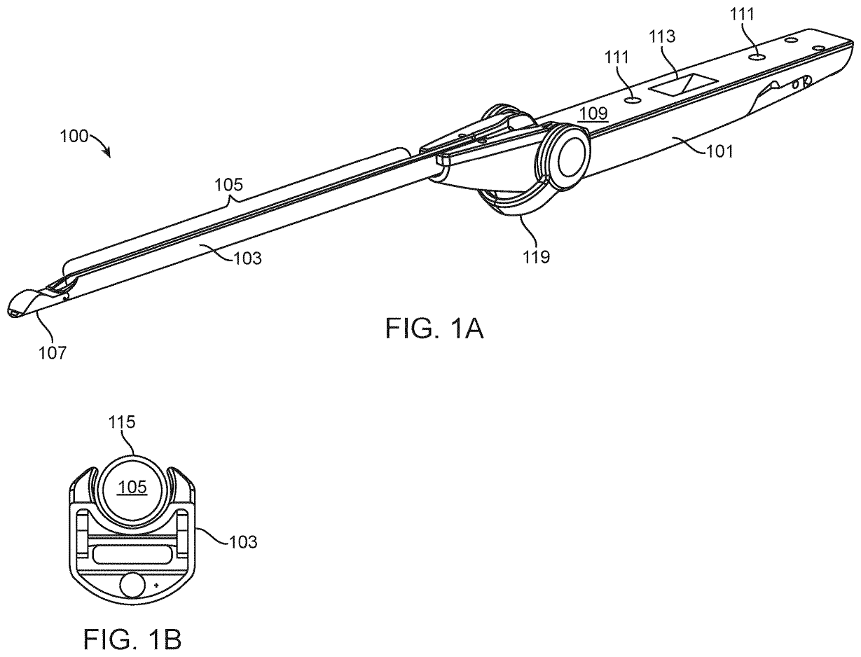

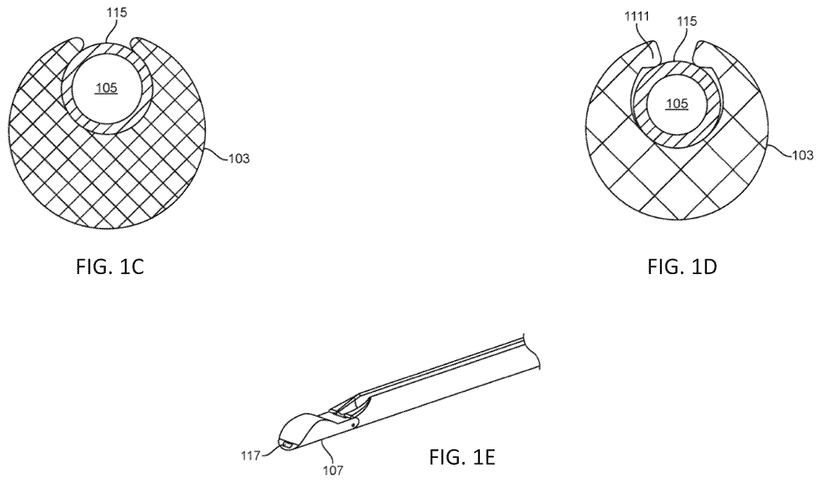

FIG. 1A shows an illustration of an imaging component 100, in accordance with some embodiments. Imaging component 100 may comprise a handle portion 101 connected to an imaging shaft 103. At the distal end of imaging shaft 103 may be coupled an imaging transducer 107. The imaging shaft 103 may comprise a proximal end and a distal end with a cavity 105 extending across the length of the shaft 103 from the proximal end towards the distal end. The cavity 105 may be at least partially open to the exterior of the shaft 103. For example, a side, or wall of the cavity 105 may comprise an elongated opening in communication with the exterior of the shaft 103. The elongated opening may be in communication with the exterior of the shaft 103 at least partially along the length of the shaft 103. In some embodiments, an edge of the elongated opening may be bent towards an interior of the cavity 105 of the shaft 103 (for example, see FIG. 1D further described below). The length of the shaft 103 may be sufficiently long to fully access the uterus of a patient while the handle portion 101 remains exterior to the patient. Additionally or alternatively, the shaft 103 may comprise a length significantly longer than the distance sufficient to fully access a patient uterus. The side opening may be open along the full length of the shaft 103 or it may be open only partially along the length of the shaft 103. The side opening may be open, for example, for greater than three-fourths the length of the shaft 103, for greater than half the length of the shaft 103, or for greater than one quarter the length of the shaft 103. The cavity 105 may be configured to receive at least one of a plurality of different additional instruments or attachments (e.g., the ablation instrument 230 of FIG. 2), such that a first instrument may be received by the cavity 105, the first instrument may be removed from the cavity 105, and a second instrument may be received by the cavity 105.

The handle portion 101 may be one part of a two-part handle such that when a first instrument or a second instrument is received the two handle portions may combine to form a single handle. The inside face of the handle portion 109 may comprise alignment elements 111 such that a first part and a second part of the handle may be reproducibly aligned with respect to one another after changing instruments. The alignment elements 111 may be configured such that a first part and a second part may be sufficiently secured with respect to one another to use the two handle portions as a single handle. In some embodiments, the alignment elements 111 may comprise magnets. In other embodiments, alignment elements 111 may comprise for example: latches, hooks, or any other mechanism suitable to removably combine a two-part handle. The handle portion may additionally comprise a positioning element 113, such as a slot to accommodate a complementary protrusion or other element on the opposite handle portion, in order to provide a more secure reference between parts of the two-part handle. The positioning element 113 may comprise a mechanical feature to secure the instrument relative to the imaging component 100 by limiting translation of the instrument on the axis of the shaft 103 of the imaging component.

In other embodiments, imaging component 100 may be configured to be used with an instrument which does not have a handle portion. In such embodiments, the handle portion 101 of the imaging component 100 is sufficient to be used alone to guide the imaging component during a procedure. In some embodiments, imaging component 100 may have a scale or a guide on the inside face of the handle portion 109 in order to gauge the insertion depth of an instrument. In other embodiments, the imaging component 100 may be used without an instrument. In some embodiments, a scale may facilitate embodiments where the instrument does not have a handle. In other embodiments, a scale may facilitate the insertion of a component of the instrument in embodiments where the instrument has a handle.

FIG. 1B shows a cross-sectional view of an imaging component 100, in accordance with some embodiments. The body of the shaft 103 may comprise internal structure in order to carry electronics or other associated components to control the imaging transducer 107. The shaft 103 may also comprise a wire system or other flex mechanism in order to allow the shaft 103 to controllably bend, flex, or deflect the distal end of the shaft 103. The shaft 103 may comprise a channel or duct to direct fluid (e.g., water, saline, etc.) to a distal end of the shaft 103 and onto a tissue surface. Imaging shaft 103 may be round in cross-section or take a shape with sufficiently softened, chamfered, rounded, or beveled edges such that the edges may be atraumatic to a patient opening during insertion or removal of an imaging component 100 with or without an instrument. Shaft 103 may additionally comprise a smooth exterior surface. Shaft 103 may be made of a material such that the surface may be deformable to allow the shaft 103 to bend or adapt to the shape of a bodily lumen.

The cavity 105 of imaging shaft 103 may be configured to slidably receive one or more of a plurality of instruments (e.g., the ablation instrument 230 of FIG. 2). In some embodiments, the cavity 105 may be defined by an exterior surface of the shaft 103. In some embodiments, the cavity 105 may be partially open along a wall, such that the cavity 105 may be in communication with the exterior of the shaft 103. The opening may be sufficiently closed to provide structural support such that when the imaging component 100 may be inserted into a patient bodily lumen, the opening of the lumen may not be significantly disturbed by the insertion or removal of an instrument. Optionally, the exterior surface of the shaft 103 may comprise only atraumatic edges. The cavity 105 of imaging shaft 103 may be sufficiently open such that when instruments of different sizes may be received or inserted into the cavity, the cavity may allow some distortion of the cavity opening. The cavity 105 may facilitate cleaning of the imaging component.

FIG. 1C shows a cross-section view of an imaging component having a shaft 103 with a circular cross-section, in accordance with some embodiments. The imaging component of FIG. 1C may be sufficiently circular in cross-section such that the imaging component may be rotated without disturbing a patient lumen. FIG. 1D shows a cross-sectional view of an imaging component with edges bent inward towards the interior of the cavity 105, in accordance with some embodiments. The inward bent edges 1111 of a cavity may serve to support the opening of a bodily lumen such that the shaft 103 may be inserted or removed atraumatically from a bodily lumen with or without an instrument.

While the cavity 105 of the shaft 103 in the illustrated example may define a circular cross sectional geometry, in other embodiments the cavity may be elliptical or any other geometric shape with sufficiently softened, rounded, or beveled edges and corners such that insertion or removal of the shaft may not damage the patient bodily lumen. In some embodiments, the cavity 105 may be non-cylindrically symmetric. In some embodiments, the cavity 105 may be asymmetrical to provide an axis for alignment of the instrument (e.g., the ablation instrument 230 of FIG. 2) within. The cavity 105 may be open for less than three-quarters its perimeter in cross-section, additionally or alternatively, the cavity may be open for less than half its perimeter, less than a quarter its perimeter, and less than one eighth its perimeter. In other embodiments, the cavity 105 of the shaft 103 of the imaging component may be closed to the exterior of the shaft, and an instrument may be slidably inserted fully interior to the shaft of the imaging component.

In some embodiments, the cavity 105 may comprise a substantially uniform cross sectional area along the shaft 103. In other embodiments, a portion of the length of the shaft 103 may have a different cross section than another portion of the length of the shaft. In an example, the proximal portion of the shaft 103 may be asymmetric to provide an axis for alignment of an instrument and the distal portion of the shaft may have a circular cross sectional area. In another embodiment, the cavity 105 tapers toward the end of the shaft 103. In such an example, the taper may facilitate feeding an instrument into the cavity 105. In some embodiments, the cross sectional area of the cavity 105 may narrow in diameter to allow greater flexibility of the distal end of the shaft 103.

In some embodiments, imaging shaft 103 may additionally comprise a tube 115 to be positioned at the cavity 105 of imaging shaft 103. Tube 115 may comprise a lumen. The lumen of tube 115 may be configured to slidably receive one or more of a plurality of instruments (e.g., the ablation instrument 230 of FIG. 2). Tube 115 may be aligned in parallel with the shaft 103 of the imaging component, such that an additional instrument/attachment may be slidably received by the tube. Subsequently, the tube 115 may slidably receive the additional instrument/attachment after it has been aligned to be in parallel with the shaft 103 of the imaging component. In some embodiments, the tube 115 may be disposable. In some embodiments, the tube 115 may be reusable such as by being un-coupled from the imaging shaft 103, washed, and autoclaved. Tube 115 may have an exterior surface wherein the surface is substantially in contact with the inner wall of cavity 105. Tube 115 may have an interior surface of a different geometry to the outer surface configured to receive one or more of a plurality of instruments. In some embodiments, a second tube (not shown) may be removably inserted into the first tube 115 and the second tube may have a different inner lumen geometry than the first, thereby aiding in the insertion of one or more of a plurality of instruments. In some embodiments, the tube 115 may be rotated relative to the imaging component. In some embodiments, the tube 115 may fully rotate relative to the imaging component in either direction under the control of a user within the shaft 103 of the imaging component. In some embodiments, the tube 115 may be internally or externally lubricated to facilitate insertion or removal of an instrument.

The tube 115 may be inserted into the bodily lumen in situ with the imaging component yet advanced therein. Additionally or alternatively, the tube 115 may be inserted into the shaft 103 of the imaging component prior to insertion of the imaging component into the bodily lumen. The tube 115 may have sufficient structural integrity to support a bodily lumen during insertion of the imaging component without an instrument. When an additional instrument (e.g., the ablation instrument 230 of FIG. 2) is inserted into the tube 115 or the tube 115 is inserted into the imaging component in situ, disruption to the bodily lumen may be minimized. The tube 115 may be made of a material that can be sterilized. The tube 115 may be made of a material that may be of low enough cost that it may be disposed of after a single use. Exemplary materials for a disposable tube may comprise polyimide, PTFE, Urethanes and thermoplastics like Pebax or Nylon, etc. Tube 115 may be made of a material comprising sufficient elasticity in order to adapt to an instrument of a size somewhat larger or smaller than the perimeter of the tube. In embodiments where the cavity 105 is not circular, the tube 115 may take the shape of the cavity or it may take another shape.

The tube 115 may lower treatment costs by facilitating insertion and/or removal of an additional instrument (e.g., the ablation instrument 230 of FIG. 2) into the cavity 105 of the imaging component 100 and thereby preventing damage to the surface of the cavity 105 of the imaging component 100. The tube 115 may lower cost by facilitating cleaning of the cavity 105 of the imaging component 100. The tube 115 may lower cost of treatment by providing an inexpensive component which may act as an adapter for a variety of different therapeutic and/or diagnostic instruments/attachments, such as being provided in a variety of different inner geometries suitable for the different instruments/attachments but having a uniform outer geometry to be removably coupled to the same single imaging component 100. For example, a disposable tube with a smaller inner diameter may facilitate the insertion and control of a needle with a smaller outer diameter than the inner diameter of the shaft 103 of the imaging component.

FIG. 1E shows a magnified view of a distal end of the imaging component 100 comprising a cavity 105, in accordance with some embodiments. The distal end of the imaging component 100 may comprise an imaging transducer 107. The imaging transducer 107 may comprise an ultrasound transducer and/or a plurality of ultrasound transducers. The ultrasound transducer may operate at a frequency of 500 kHz, 1 MHZ, 5 MHz, 10 MHz, 20 MHz, 100 MHz, or a range defined by any two of the preceding values. Some embodiments of the ultrasound transducer may comprise specifications of other transducers from the commonly assigned references incorporated herein.

In some embodiments, the distal end 117 of the imaging transducer 107 may additionally comprise a light emitting diode and/or a camera in order to provide images to a user. In such embodiments, the imaging component 100 may serve as an optical scope as well as an ultrasound imaging platform. The distal end 117 of the imaging transducer 107 may comprise optical components, such as an optic fiber, a relay lens, an objective lens, etc.

The imaging transducer 107 may be configured to be deflectable. The imaging transducer 107 may be configured to deflect relative to the longitudinal axis of the shaft 103 of the imaging component 100. In some embodiments, the distal end of an imaging component 100 comprises a hinge to facilitate deflection of an imaging transducer 107. The deflection of the imaging transducer 107 may be controlled by a deflection lever 119 on the handle portion 101 of the imaging component 100. The one or a plurality of imaging transducers 107 may be oriented by the deflection of the imaging transducer. The one or a plurality of imaging transducers 107 may be oriented by the deflection of the imaging transducer in order to facilitate maintaining the field of view of an image during a treatment. Additionally or alternatively, the imaging transducers 107 (e.g., ultrasound transducers) may be aligned radially and/or axially to image multiple views simultaneously. Deflection of the imaging transducer 107 may be induced in order to avoid obstruction of an instrument (e.g., the ablation instrument 230 of FIG. 2). Additionally or alternatively, deflection of the imaging transducer 107 may be used to deflect a flexible instrument within the cavity 105. The distal end of the shaft 103 may comprise an interlock system, similar to those in the incorporated references, in order to prevent the imaging transducer 107 from obstructing an instrument or being damaged by sharp edges of an instrument. Actuation of the deflection lever 119 may function in a manner similar to that described in U.S. Pat. No. 8,992,427, incorporated herein by reference. The deflection lever 119 may deflect the imaging transducer 107 by less than 45 degrees and additionally or alternatively, for example, less than 120 degrees, less than 90 degrees, less than 60 degrees, less than 30 degrees, less than 15 degrees, and less than 5 degrees.

The distal end of the imaging component 100 may comprise atraumatic edges in order to facilitate insertion of the imaging component with or without an instrument in the cavity 105. The distal end of the cavity 105 of the imaging component 100 may additionally or alternatively comprise a portion angled axially relative to the shaft 103, such that a distal end of an instrument may be deflected upward as it is pushed out the distal end of the cavity 105. The distal end of the cavity 105 of the imaging component 100 may comprise an angled portion with an angle of 3 to 45 degrees. The distal end of the cavity 105 of the imaging component 100 may comprise an angled portion with an angle at less than 45 degrees and additionally or alternatively, for example, less than 90 degrees, less than 60 degrees, less than 30 degrees, less than 15 degrees, and less than 5 degrees.

The cavity 105 of the imaging component 100 may be configured to slidably receive one or more of a plurality of instruments (e.g., the ablation instrument 230 of FIG. 2). In some embodiments, the imaging component 100 may be configured to receive one or a plurality of therapeutic or diagnostic instruments. In some embodiments, at least one of the plurality of different instrument may be a therapeutic or diagnostic instrument. In some embodiments, the instrument may comprise an instrument such as therapy electrodes; diagnostic electrodes and/or needles; a tissue ablation element, such as for example a radiofrequency ablation element, an ultrasonic ablation element, a heat-based ablation element, a cryoablation element, etc.; and/or other instrument suitable to be disposed within the cavity of the imaging component. Additionally or alternatively, the instrument may be used to deliver drugs or other therapeutic agents to the tissue to be treated. FIG. 2 shows an ablation instrument 230 which may be slidably received by the imaging component. One of ordinary skill in the art will recognize that many instruments, including those disclosed in the FIG. 2, may be used with the imaging component disclosed herein.

FIG. 2 shows a magnified view of a distal end of the imaging component 100 with an ablation instrument 230 disposed within the shaft 103 of the imaging component 100, in accordance with some embodiments. The ablation instrument 230 may contain a needle assembly comprising an introducer 235 and, optionally, electrode ablation needles, or tines 233. The shaft 231 of the ablation instrument 230 may be deployed from the shaft 103 of an imaging component 100. Additionally or alternatively, the introducer 235 may be deployed from a lumen of a tube 115. The ablation instrument 230 may comprise one or more of, for example, a radiofrequency (RF) ablation element, an ultrasonic ablation element, a heat-based ablation element, a cryoablation element, and any other type of ablation elements known to one of ordinary skill in the art.

Ablation instrument 230 may be disposed within a tube 115 disposed within the cavity 105 of the imaging component 100. Additionally or alternatively, ablation instrument 230 may be disposed within the cavity 105 of the imaging component 100 without the use of a tube. While the shaft 231 of the ablation instrument 230 in the illustrated example may define a circular cross-sectional geometry, in other embodiments, the shaft 231 of the ablation instrument 230 may be elliptical or any other geometric shape such that the shaft 231 may be inserted or removed from the cavity 105 of the imaging component 100. In some embodiments, the shaft 231 of the ablation instrument 230 may be asymmetrical to provide an axis for alignment of the instrument within the cavity 105 of the imaging component 100.

The shaft 231 of the ablation instrument 230 may be made of a pliable and/or flexible material such that it may be deflected by the imaging transducer 107 and/or an angled portion within the cavity 105 of the shaft 103 of the imaging component 100. The distal end of a shaft 231 of the ablation instrument 230 may be deflected up in order to avoid damage of the imaging transducer 107, among other possible purposes. The distal end of the cavity 105 of the imaging component 100 may comprise a portion angled axially relative to the shaft 103, such that a distal end of an instrument (e.g., the ablation instrument 230) may be deflected upward as it is pushed out the distal end of the cavity 105. The distal end of the cavity 105 of the imaging component 100 may comprise an angled portion angled less than 45 degrees and additionally or alternatively, for example, less than 90 degrees, less than 60 degrees, less than 30 degrees, less than 15 degrees, and less than 5 degrees.

Additionally or alternatively, the shaft 231 of the ablation instrument 230 may comprise a wire system or other means to deflect the distal end of the ablation instrument 230 such that a distal end of the ablation instrument 230 does not damage the imaging transducer 107. In some embodiments, the ablation instrument 230 may rotate relative to the imaging component 100. In some embodiments, the ablation instrument 230 may fully rotate relative to the imaging component 100 in either direction under the control of a user within the shaft 103 of the imaging component 100 while the shaft 103 remains stationary, such that the tines 233 may be optimally aligned.

The needle assembly may be constructed and controlled by a user, for example, as previously described in commonly owned U.S. Pat. Nos. 8,206,300, 8,262,574, and 8,992,427, the full disclosures of which are incorporated herein by reference. The needle assembly may be integrated into an instrument handle such that the position and deployment of the introducer 235 and plurality of electrode ablation needles or tines 233 may be controlled by the user. The handle may be constructed, for example, as previously described in commonly owned U.S. Pat. No. 8,992,427, the full disclosure of which is incorporated herein by reference. The needle assembly may be compatible with systems and methods for improved safety and treatment boundaries during the treatment of uterine fibroids as, for example, described in the incorporated references. FIG. 2 illustrates an exemplary instrument which may be disposed within the shaft 103 of an imaging component 100, which example is not intended to be limiting.

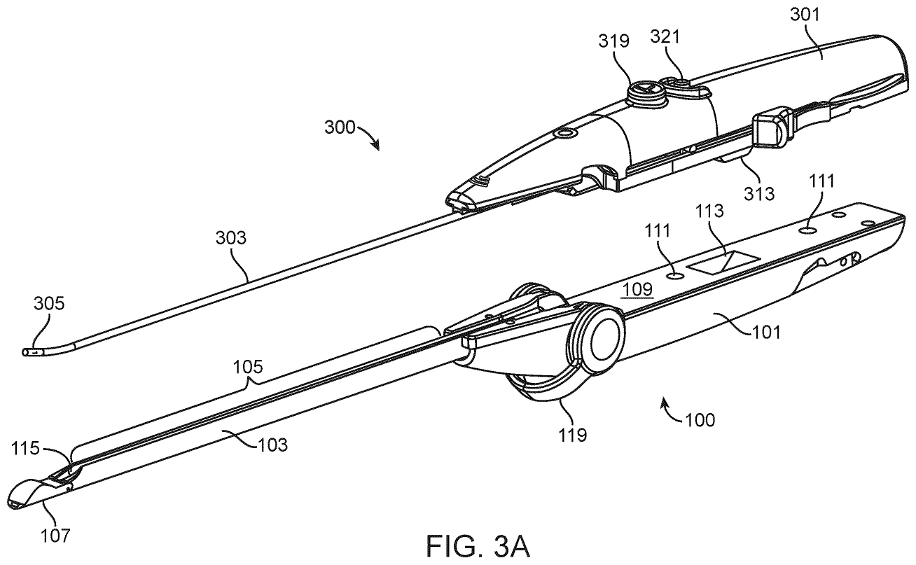

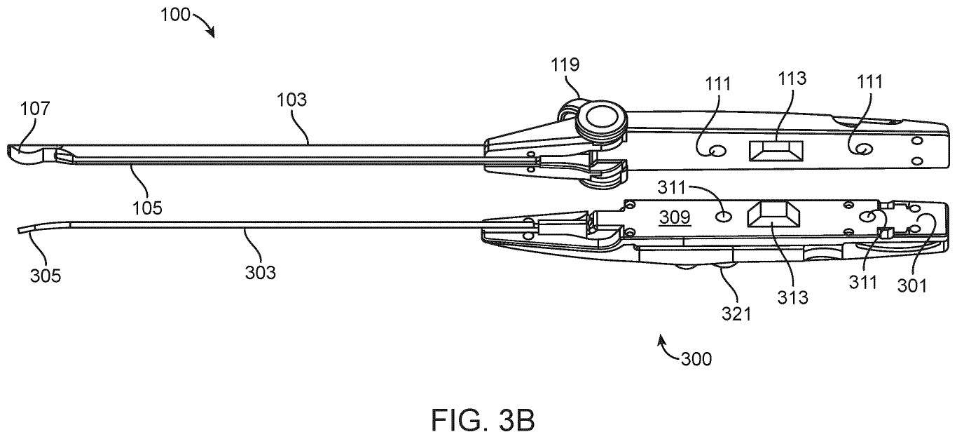

FIG. 3A shows an assembly view of an imaging system comprising an imaging component 100 and an optical scope instrument 300, in accordance with some embodiments. While an optical scope element may be shown in the illustrated embodiment, optical scope instrument 300 may be any other suitable instrument, for example, any of the instruments disclosed herein (e.g., the ablation instrument 230 of FIG. 2). Illustrated in FIG. 3A, the imaging system may slidably receive a disposable tube 115 within the cavity 105 of the imaging component 100. In some embodiments, the imaging system may comprise a disposable tube 115 slidably received within the cavity 105 of the imaging component 100. In such embodiments, an instrument may be removably received with a lumen of the disposable tube 115. Additionally or alternatively, the cavity 105 of the imaging component 100 may be configured to slidably receive one or more of a plurality of instruments, which instruments may comprise various therapeutic and/or diagnostic instruments.

In illustrative examples, the imaging component may removably receive an instrument such as therapy electrodes; diagnostic electrodes and/or needles; a tissue ablation element, such as for example a radiofrequency ablation element, an ultrasonic ablation element, a heat-based ablation element, a cryoablation element, etc.; and/or other instrument suitable to be disposed within the cavity of the imaging component. Additionally or alternatively, the instrument may be used to deliver drugs or other therapeutic agents to the tissue to be treated. Additionally or alternatively, with or without the use of a disposable tube, the imaging component may removably receive the ablation instrument 230 illustrated in FIG. 2.

In the illustrated embodiment, the distal end 305 of the optical scope instrument 300 may comprise a light emitting diode and/or a camera in order to provide images to a user. In such embodiments, the optical scope instrument 300 may serve as an endoscope. The distal end 305 of the optical scope instrument 300 may comprise optical components, such as an optic fiber, a relay lens, an objective lens, etc. The optical scope instrument 300 may comprise a shaft 303 of an optical scope instrument 300, which has a distal end and a proximal end. The shaft 303 of optical scope instrument 300 may be configured to detach from a handle component of the instrument or may be configured to be used without a handle component such that the optical scope instrument 300 may be disposable.

The shaft 303 of optical scope instrument 300 may be made of a pliable and/or flexible material such that it may be deflected by the imaging transducer 107 and/or an angled portion within the cavity 105 of the shaft 103 of the imaging component 100. Additionally or alternatively, the shaft 303 of the optical scope instrument 300 may comprise a (e.g., push, pull, and/or rotate/torque) wire system or other means to deflect the distal end 305 of the optical scope instrument 300. Deflection of a distal end 305 of an optical scope instrument 300 may serve to prevent damage to the imaging transducer 107 and/or allow multiple image angles may be collected. In some embodiments, the optical scope instrument 300 may rotate relative to the imaging component 100. In some embodiments, the optical scope instrument 300 may fully rotate relative to the imaging component 100 in either direction under the control of a user within the shaft 103 of the imaging component 100 while the shaft 103 of the imaging component remains stationary, such that multiple image angles may be collected.

The shaft 303 of the optical scope instrument 300 may be longer than the shaft of the imaging transducer 107 such that images may be collected from deep inside the uterus. In some embodiments, the shaft 303 of the optical scope instrument 300 may be two inches longer than the shaft of the imaging transducer 107. Additionally or alternatively, for example, the shaft 303 may be six inches longer, may be four inches longer, may be two inches longer, may be the same length, or may be within a range of any two of the preceding values.

In the illustrated embodiment, the optical scope instrument 300 comprises a handle portion 301. While a handle portion 301 may be shown connected to an optical scope in the illustrated example, similar handle portions may be connected to any suitable instrument (e.g., the ablation instrument 230 of FIG. 2), such as those disclosed herein. The handle portion 301 may be the second part of a two-part handle such that when an optical scope instrument 300 may be slidably inserted into the imaging component 100, the two handle portions may combine to form a single handle. The handle portion may additionally comprise a positioning element 313, in order to provide a more secure reference between parts of the two-part handle. Positioning element 313 may mate with positioning element 113. In such embodiments, the handle portion may comprise a release control 321, which may be actuated by a user, to retract the positioning element 313 into the handle and allow the two-part handle to be separated.

The handle portion 301 may additionally comprise one or a plurality of control elements 319. Control elements 319 may allow a medical professional to control the distal end of an instrument (e.g., the ablation instrument 230 of FIG. 2). In one example, the control element 319 controls a wire system which may reproducibly deflect or steer a distal end of an instrument. Additionally or alternatively, the control element 319 rotates a shaft of an instrument (e.g., a shaft 303 of an optical scope instrument 300) within the cavity 105 of the imaging component 100 or within the disposable tube 115. In another example, the control element scoops tissue in a tissue collector instrument. In another example, the control element 319 deploys a needle assembly comprising optional tines in an ablation instrument. Additionally or alternatively, the control element 319 begins the ablation procedure. In another example, the control element 319 applies pressure to inject a chemical though a drug delivery instrument. In another example, the control element 319 begins or ends image collection in an optical scope instrument.

FIG. 3B shows an assembly view of an imaging system illustrating an attachment mechanism of a system, in accordance with some embodiments. The inside 309 of the handle portion 301 may comprise alignment elements 311. Alignment elements 311 may be configured such that the optical scope instrument 300 may be reproducibly aligned with respect to the imaging component 100 after changing instruments. Additionally or alternatively, the alignment elements 311 may sufficiently secure the instrument (e.g., the ablation instrument 230 of FIG. 2) and the imaging component 100 with respect to one another to use the two handle portions 101, 301 as a single handle. In some embodiments, the alignment elements 311 may comprise magnets. In other embodiments, the alignment elements 311 may comprise for example: latches, hooks, or any other mechanism suitable to removably combine a two-part handle. The inside 309 of the handle portion 301 may additionally comprise a positioning element 313, in order to provide a more secure reference between parts of the two-part handle. In such embodiments, the handle portion may comprise a release control 321, which may be actuated by a user, to retract the positioning element 313 into the handle and allow the two-part handle to be separated.

In some embodiments, a method of detecting or sensing the identification of removable instruments is provided when coupling the imaging component 100 and the removable instrument (e.g., the optical scope instrument 300). The imaging component 100 may include software to recognize the removable instrument and manage the interconnection between the imaging component 100 and removable instrument. The sensor or mechanism may be, by way of non-limiting examples, optical, RF, magnetic, biometric, electronic and mechanical IDs and readers. The method will ensure only qualified removable devices are received on the imaging device to ensure that only compatible devices may be used with the imaging component 100.

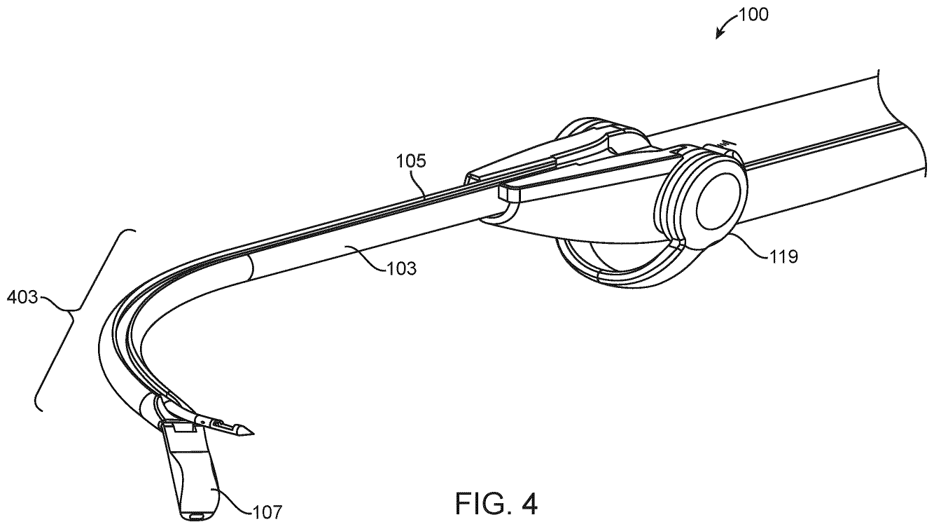

FIG. 4 illustrates a shaft 103 of an imaging component 100 wherein the shaft 103 of the imaging component 100 may be flexible, in accordance with some embodiments. In the illustrated embodiment, the shaft 103 of the imaging component 100 may comprise a flexible shaft portion 403. The body of the flexible shaft portion 403 may comprise internal structure in order to carry electronics or other associated components to control the imaging transducer 107. The imaging transducer 107 may comprise a channel or duct to direct fluid (e.g., water, saline, etc.) to a distal end of the shaft and onto a tissue surface. The flexible shaft portion 403 may comprise a fraction of the length of the shaft 103 of the imaging component 100. In some embodiments, the flexible shaft portion 403 comprises less than three-quarters the length of the shaft 103. Additionally or alternatively, the flexible shaft portion 403 may comprise less than a quarter the length of the shaft 103, and less than one eighth the length of the shaft 103, and the full length of the shaft 103.

The cross-sectional geometry of the flexible shaft portion 403 may continue the geometry of the shaft 103 such that no gaps or traumatic edges may be created between the flexible shaft portion 403 and the shaft 103. The flexible shaft portion 403 may be round in cross-section or take a shape with sufficiently softened, chamfered, rounded or beveled edges such that the edges may be atraumatic to a patient opening during insertion or removal of an imaging component 100 with or without an instrument. The flexible shaft portion 403 may additionally comprise a smooth exterior surface. The flexible shaft portion 403 may be made of a material such that the surface may be deformable to allow the flexible shaft portion 403 to bend or adapt to the shape of a bodily lumen.

The cavity of the flexible shaft portion 403 may be configured to slidably receive one or more of a plurality of instruments. The cavity of the flexible shaft portion 403 may be configured to continue the shape of the cavity 105 of the shaft 103 such that no gaps or traumatic edges may be created between the flexible shaft portion 403 and the shaft 103. In some embodiments, the cavity of the flexible shaft portion 403 may be partially open along a wall, such that a lumen of the cavity of the flexible shaft portion 403 may be in communication with the exterior of the shaft 103. The opening of the flexible shaft portion 403 may be sufficiently closed to provide structural support such that when the imaging component 100 may be inserted into a patient bodily lumen, the opening of the lumen may not be significantly disturbed by the insertion or removal of an instrument. In some embodiments, the edges of a cavity of the flexible shaft portion 403 may bend inward towards the interior of the cavity, such as in the embodiment illustrated in FIG. 1D. The inward bent edges of a cavity of the flexible shaft portion 403 may serve to support the opening of a bodily lumen such that the shaft 103 may be inserted or removed atraumatically from a bodily lumen with or without an instrument. The cavity of the flexible shaft portion 403 may be sufficiently open such that when instruments of different sizes may be received or inserted into the cavity, some distortion of the cavity opening may occur. The cavity may facilitate cleaning of the imaging component 100 by providing access to the interior of the cavity from its exterior.

While the cavity of the flexible shaft portion 403 in the illustrated example defines a circular cross sectional geometry, in other embodiments the cavity of the flexible shaft portion 403 may be elliptical or any other geometric shape with sufficiently softened, rounded, or beveled edges and corners such that insertion or removal of the shaft of the flexible shaft portion 403 does not damage the patient bodily lumen. In some embodiments, the cavity of the flexible shaft portion 403 may be asymmetrical to provide an axis for alignment of the instrument within. The cavity of the flexible shaft portion 403 may be open for less than three-quarters of its perimeter in cross-section, additionally or alternative, the cavity may be open for less than half its perimeter, less than a quarter its perimeter, and less than one eighth its perimeter. In other embodiments, the cavity of the flexible shaft portion 403 may be closed to the exterior of the shaft of the flexible portion, and an instrument may be slidably inserted fully interior to the shaft of the flexible portion.

In some embodiments, the flexible shaft portion 403 may be constructed from a pliable and/or flexible material such that it may be flexed within a patient bodily lumen. In some embodiments, the shaft may be controllably flexed along its longitudinal axis via a flex mechanism. Additionally or alternatively, the flexible shaft portion 403 may comprise a wire system or other flex mechanism in order to allow the flexible shaft portion 403 to controllably bend, flex, or deflect the distal end of the flexible portion. The flex mechanism may be controlled by a control element on a handle portion (e.g., handle portion 101, shown in FIG. 3A) of the imaging component 100.

In the illustrated example, the flexible shaft portion may be flexed axially to about a 90 degree angle with respect to the handle. Additionally or alternatively, the flexible shaft portion may be flexed axially to, for example, less than 180 degrees, less than 120 degrees, less than 90 degrees, less than 45 degrees, less than 10 degrees, less than 1 degrees. Additionally or alternatively, the flexible shaft portion may be flexed in an anterior-posterior axis relative to the handle of the imaging component 100. In some embodiments, the flexible shaft portion may be flexed in an anterior-posterior axis to, for example, less than 180 degrees, less than 120 degrees, less than 90 degrees, less than 45 degrees, less than 10 degrees, less than 1 degrees. Additionally or alternatively, the flexible shaft portion may be flexed in a medial-lateral axis relative the handle of the imaging component 100. In some embodiments, the flexible shaft portion may be flexed in a medial-lateral axis to, for example, less than 180 degrees, less than 120 degrees, less than 90 degrees, less than 45 degrees, less than 10 degrees, less than 1 degrees.

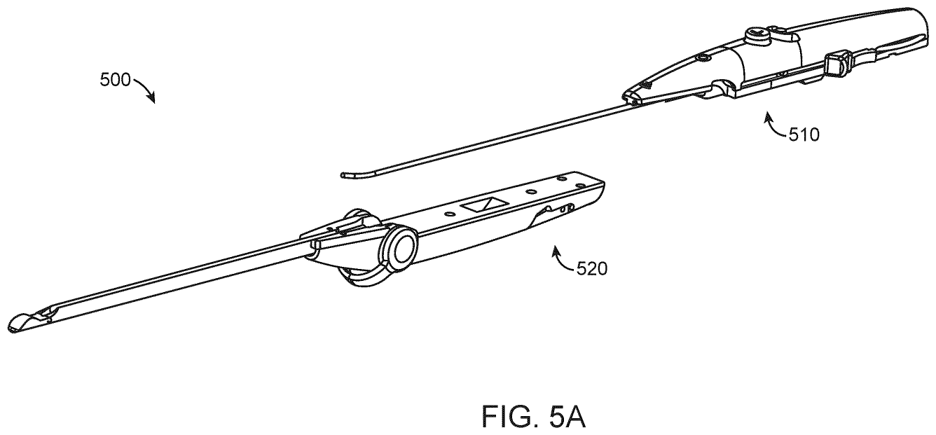

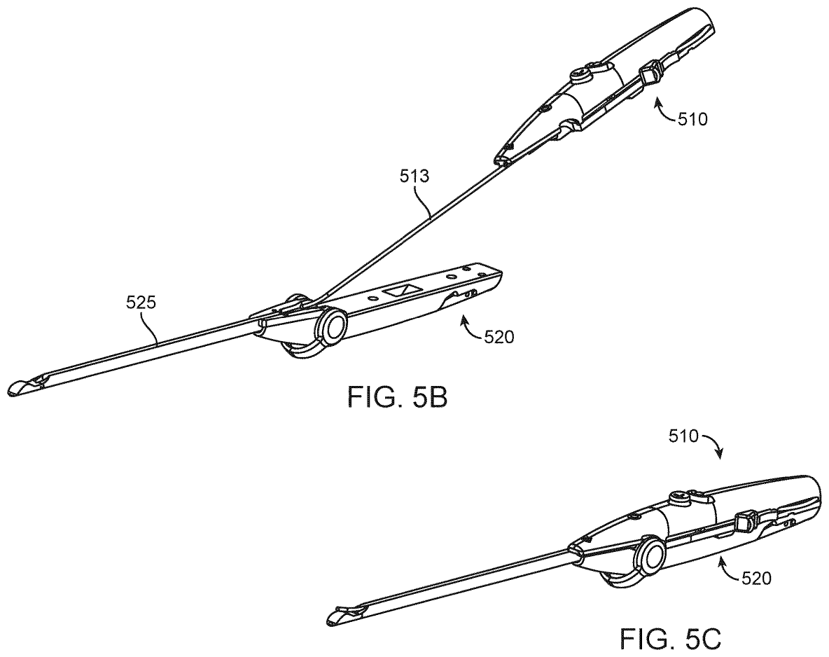

FIG. 5A illustrates a system for diagnosing and/or providing therapy, which may be removably coupled to multiple therapeutic and/or diagnostic instruments (e.g., the ablation instrument 230 of FIG. 2), in accordance with some embodiments. A system for performing therapy and/or diagnosis may comprise a therapeutic or diagnostic instrument 510 and an imaging component 520. An instrument 510 of the system for performing therapy and/or diagnosis may comprise a therapeutic or diagnostic instrument, such as, for example, any of the therapeutic or diagnostic instruments described herein (e.g., the ablation instrument 230 of FIG. 2). In some embodiments, the imaging component 520 may be used in conjunction with an instrument such as therapy electrodes; diagnostic electrodes and/or needles; a tissue ablation element, such as for example a radiofrequency ablation element, an ultrasonic ablation element, a heat-based ablation element, a cryoablation element, etc.; and/or any other instrument suitable to be disposed within the cavity of the imaging component. Additionally or alternatively, the instrument may be used to deliver drugs or other therapeutic agents to the tissue to be treated. FIG. 2 shows exemplary instruments which may be slidably received by the imaging component. In some embodiments, the system may comprise a first and a second therapeutic or diagnostic instrument. An imaging component 520 may comprise an imaging component, such as, for example, examples, embodiments, and variations on the imaging component described herein.

FIG. 5B illustrates a system for diagnosing and/or providing therapy with a therapeutic and/or diagnostic instrument 510 (e.g., the ablation instrument 230 of FIG. 2) being removably coupled to an imaging component 520, in accordance with some embodiments. As shown, the instrument 510 may be axially aligned with respect to the imaging component 520. Additionally, the distal end of the shaft 513 of the instrument 510 may be fed into the proximal end of the cavity 525 of the imaging component 520. Subsequently, the instrument 510 may be advanced toward the imaging component 520, such that the shaft 513 of the instrument 510 is slidably received by the cavity 525 of the imaging component 520. The instrument 510 may be slidably removed from the imaging component 520 by a similar procedure.

FIG. 5C illustrates a system for diagnosing and/or providing therapy with a therapeutic and/or diagnostic instrument 510 removably coupled to an imaging component 520, in accordance with some embodiments. The system for diagnosing therapy may comprise retention elements such as hooks, latches, or the mechanical features described herein in order to secure the instrument 510 to the imaging component 520. The system for diagnosing and/or providing therapy may be configured to couple to a plurality of instruments. For example, a first instrument may be coupled to an imaging component 520, and, subsequently, a second instrument may be coupled. The imaging component 520 may be configured to be coupled to both the first and second therapeutic and/or diagnostic instrument either simultaneously or individually. For example, if the first instrument is a disposable tube, the second instrument may be slidably inserted within the first instrument. In some embodiments, the imaging component 520 may be configured to be deliverable to the target site within the patient previously coupled with the first and/or second therapeutic or diagnostic instruments exterior to the target site. Additionally or alternatively, the imaging component 520 may be configured to be removably coupled to both the first and second therapeutic or diagnostic instruments, either simultaneously or individually, after the imaging component 520 is delivered to the target site within the patient (e.g., the instrument may be coupled in situ).

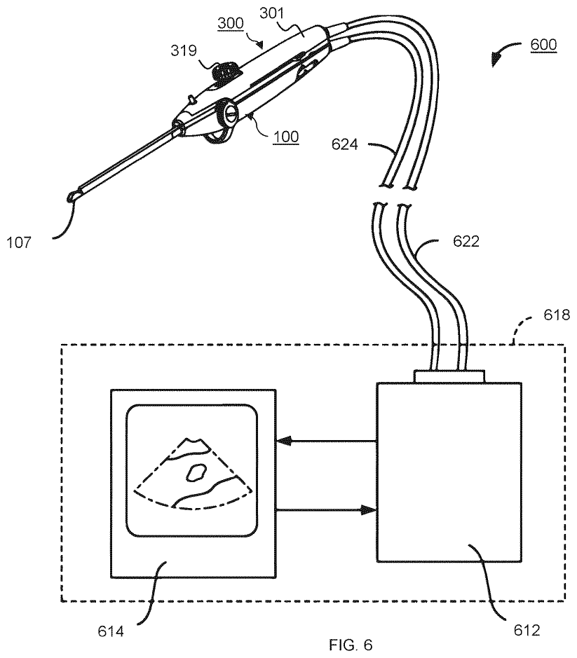

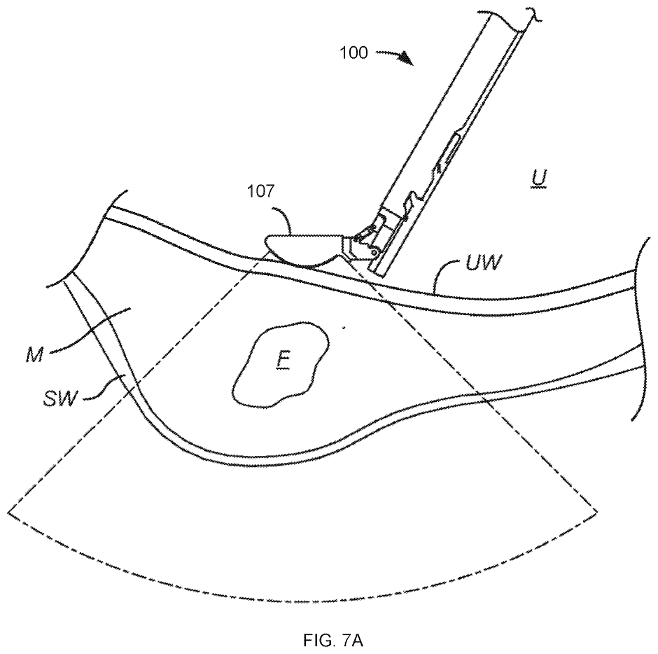

FIG. 6 shows a system 600 which may include a system controller 612, an imaging display 614, and a treatment probe, which in FIG. 6 comprises the two attached subcomponents of an imaging component 100 and an instrument 300. It will be understood by a skilled artisan that the instrument 300 of the treatment probe can be an ablation instrument (e.g., the ablation instrument 230, shown in FIG. 2). The system controller 612 will typically be a microprocessor-based controller which allows both treatment parameters and imaging parameters to be set in a conventional manner. The display 614 will usually be included in a common enclosure 618 together with the controller 612, but could be provided in a separate enclosure. The treatment probe 100, 300 may include an imaging transducer 107 which may be connected to the controller 612 by an imaging cord 624 to provide the image(s) captured by the imaging component 100 to the controller 612 to be displayed by the display 614; however, additionally or alternatively, the imaging component 100 may communicate with the controller 612 wirelessly. The instrument 300 may be connected to and in communication with the controller 612 via an instrument cord 622 such as to provide one or more of a control signal, a feedback signal, a position signal, or a status signal; however, additionally or alternatively, the instrument 300 may communicate with the controller 612 wirelessly. In embodiments where the imaging component 100 and the instrument 300 are connected by cords 624, 622, the controller 612 may supply power to either or both components.

The controller 612 will typically further include an interface for the treating physician to provide information to the controller 612, such as a keyboard, touch screen, control panel, mouse, joystick, directional pad (i.e., a D-pad), or the like. Optionally, a touch panel may be part of the imaging display 614. The energy delivered to the treatment probe 100, 300 by the controller 612 may be radiofrequency (RF) energy, microwave energy, a treatment plasma, heat, cold (cryogenic therapy), or any other conventional energy-mediated treatment modality. Alternatively or additionally, the treatment probe 100, 300 could be adapted to deliver drugs or other therapeutic agents to the tissue anatomy to be treated. In some embodiments, probe 100, 300 plugs into an ultrasound system and into a separate radio frequency (RF) generator. An interface line connects the ultrasound system and the RF generator. In some embodiments in which the instrument 300 is an ablation instrument (e.g., the ablation instrument 230, shown in FIG. 2), the RF generator is configured to deliver energy to the needle assembly, which can then be used to ablate the target tissue.

The instrument 300 may comprise a handle portion 301 having one or more slidably mounted control elements 319 on its upper surface. In some embodiments, the control elements 319 may control the positioning of internal stops within the handle which may be monitored by the controller 612 in order to calculate the size and position of the boundaries of the targeting region and/or the safety region which are shown on the display 614. In embodiments where instrument 300 is an ablation instrument (e.g., ablation instrument 230, shown in FIG. 2), the stops may also serve to physically limit deployment of the introducer and optionally electrode ablation needles or tines.

Some embodiments of the methods and systems of the present disclosure may be integrated with systems and methods for establishing and adjusting displayed safety and treatment zone boundaries. Such embodiments may include systems and methods of the incorporated references including: U.S. Pat. Pub. No. 2014/0073910 (now U.S. Pat. No. 9,861,336; US. Pat. No. 8,992,427; U.S. Pat. No. 11,219,483; and P.C.T. Pub. No. WO2018/089523, the contents of which are incorporated herein by reference. Some embodiments of the methods and systems of the present disclosure may be integrated with systems and methods for mapping and planning systems. Such embodiments may include systems and methods of the incorporated references including P.C.T. Pub. No. WO2018/089523.

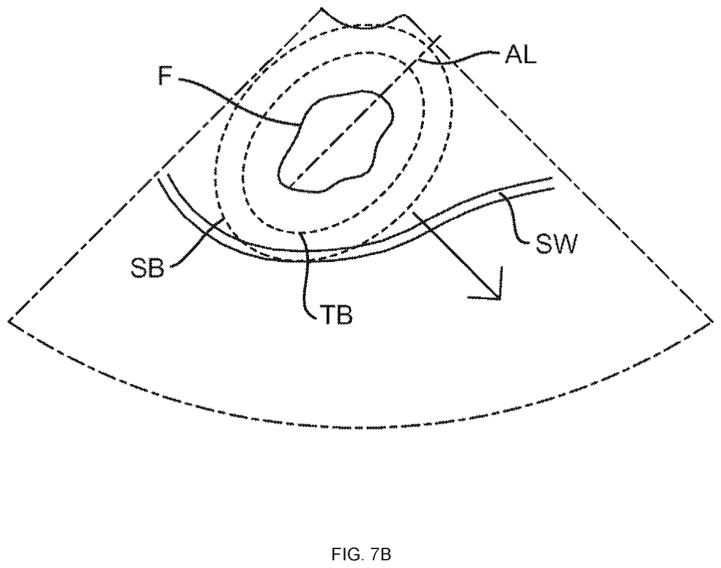

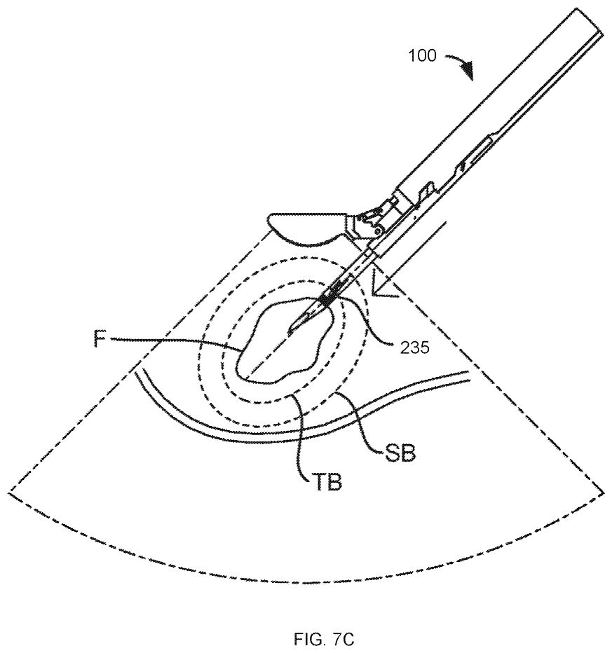

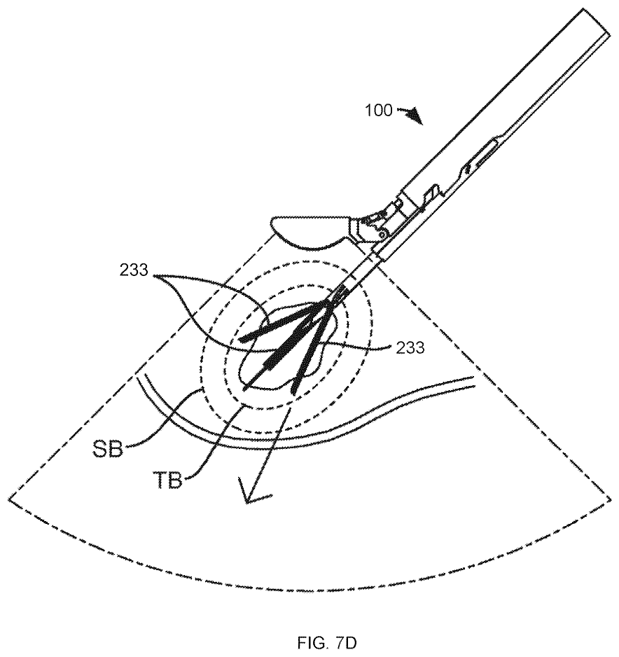

FIG. 7A illustrates an imaging component 100 which may be used to treat a fibroid F located in the myometrium M in a uterus U beneath a uterine wall UW (the endometrium) and surrounded by the serosal wall SW. The imaging component 100 can be introduced transvaginally and transcervically (or alternately laparoscopically) to the uterus U, and the imaging transducer 107 deployed to image the fibroid F within a field of view indicated by the broken lines. The needle assembly is in its retracted position and so is not shown in FIG. 7A

FIG. 7B shows an image that would be visible on a display (e.g. display 614, shown in FIG. 6), showing safety and treatment boundaries, in accordance with some embodiments. In some embodiments, once the fibroid F is located on the display 614, the controls on the handle may be used to locate and size both a treatment boundary TB and a safety boundary SB. In some embodiments, initially, the virtual boundary lines TB and SB may neither be positioned over the fibroid F nor properly sized to treat the fibroid F. Prior to beginning therapy, the user (e.g., a physician) may want to both position and size the boundaries TB and SB for proper treatment. As the imaging transducer 107 may be already positioned against the uterine wall UW the only way to advance the treatment and safety boundaries TB, SB may be to move the boundaries forward by actuating the control element 319. In some embodiments, this may cause the treatment and safety boundaries TB and SB to move forwardly along the axis line AL and thereby translate the area to be treated. This may cause the virtual boundaries on the real-time image display 614 to move over the image of the fibroid F. Additionally or alternatively, the size of the treatment boundary TB may be enlarged or shrunk in order to mitigate the risk of affecting healthy and/or more sensitive tissue around the area of treatment.

In embodiments where the instrument is a tissue ablation element, while holding imaging component 100 steady, the physician may then advance a needle slide, causing the introducer 235 to extend into the fibroid F, as shown in FIG. 7C. The introducer 235 is shown in its deployed or extended position; the plurality of electrode ablation needles or tines is in its retracted position within the introducer and so is not shown in FIG. 7C. The illustration in FIG. 7C includes a representation of the imaging component 100, which corresponds to the physical probe which is present in the patient. The remainder of FIG. 7C corresponds to the image present on the target display 614.

After the introducer 235 has been fully deployed as limited by an optional physical or virtual needle stop housing in the instrument handle 301, the electrode ablation needles or tines 233 may be deployed by advancing a tine slide. A target level of tine deployment is reached as indicated by engagement of the tine slide with an optional tine stop or visually on the display 614. Optionally, the imaging component 100 may be rotated about a central axis (typically aligned with the axis of the introducer 235) to confirm the treatment and safety boundaries TB, SB in all planes of view about the fibroid F. Display 614 will show the position of the treatment and safety boundaries TB, SB in real time relative to the target fibroid F and serosa. The plurality of electrode ablation needles or tines 233 are then configured as shown in FIG. 7D, and power can be supplied to the tines 233 (and optionally to the introducer 235) in order to achieve treatment within the boundary depicted by the virtual treatment boundary TB. In FIG. 7D, both the introducer 235 (see FIG. 7C) and plurality of electrode ablation needles or tines 233 are shown in their deployed or extended positions. It will be understood that in some ablation procedures (e.g., those in which the target tissue is small in size), the introducer 235 and/or plurality of electrode ablation needles or tines 233 will be only partially deployed. It will also be understood that after a procedure is complete, the introducer 235 and plurality of electrode ablation needles or tines 233 can be retracted from a fully or partially deployed position to a retracted position. Again, FIG. 7D mixes both the virtual image which would be present on the display 614 as well as the physical presence of the imaging component 100.

### Uterine Fibroid Cutting and Ablation Examples

As disclosed above and shown in FIG. 6, uterine fibroid ablation systems and devices may use RF or other energy to coagulate (e.g., to ablate) uterine fibroids or other tissue. According to embodiments of the present disclosure, in addition to ablating uterine fibroids or other tissue, embodiments of such systems and devices may include a cutting or insertion mode to the treatment procedure which will assist the ablation element, e.g., (RF or other energy) introducer and electrodes, in penetrating into the fibroid or tissue more easily and more accurately.

Some fibroids have a hard, capsulated surface layer or dense structures which frequently cause difficulties with tissue penetration. For instance, such a surface layer may provide resistance to penetration of ablation needles and the like, cause deflection of such needles when advanced toward the fibroid, and/or cause deflection of such the path of such needles during introducer deployment. These may prolong the process of positioning the ablation device and/or the deployment of its electrode, or otherwise cause inaccuracy of the tissue targeting and cause procedural delay. Aspects of the present disclosure may soften the fibroid surface layer and/or dense tissue, reducing the resistance to the introducer tip/electrode(s) and can improve treatment accuracy and efficiency.

According to aspects of the present disclosure, a system for penetrating target tissue can comprise an ablation element and a radiofrequency (RF) generator configured to deliver energy to the ablation element. In some embodiments, the system for penetrating target tissue is also a system for ablating target tissue. In some embodiments, the target tissue can be a fibroid, such as a uterine fibroid. In some embodiments, the system can further include an ultrasonic imaging device. And in some embodiments, the system can also include a controller that is designed to control the delivery of energy to the ablation element.

The ablation element can be configured to penetrate the target tissue. The ablation element can be further configured to ablate the target tissue. The ablation element can comprise, e.g., the needle assembly shown in FIG. 2, which can further comprise an introducer 235 and, optionally, electrode ablation needles, or tines 233. As shown in FIGS. 7A, 7C, and 7D, in some embodiments, the ablation elements are first inserted into the target tissue, then second, used to ablate the target tissue.

The RF generator can be configured to deliver energy to the ablation element. The RF generator can be a monopolar RF generator. In some embodiments, the RF generator can be a bipolar RF generator. In monopolar mode, the output features from the RF generator can be essential in determining the particular extensiveness of the impact on tissue and the power with which instruments perform. Within the monopolar circuit, there is typically an active electrode in the surgical site and a return electrode in a distant site that is generally positioned on the thigh of the patient. The current may flow through the body between the electrodes.

According to aspects the present disclosure, the RF generator can have a plurality of different modes or settings configured for different steps of a procedure. For example, according to the present disclosure, the RF generator may comprise three waveform settings: cut, blend, and coagulate/ablate. The blend mode and the coagulate/ablate mode are optional. While the cut mode and coagulate/ablate mode are detailed below, it will be understood by a skilled artisan that the blend mode provides energy in a manner between the cut mode and the coagulate/ablate mode. The cut mode may also be referred to herein as an insertion mode. In some embodiments, the tissue in the cut mode or insertion mode may not actually be cut. In some embodiments, in the cut mode or insertion mode, the tissue is softened to facilitate penetration.

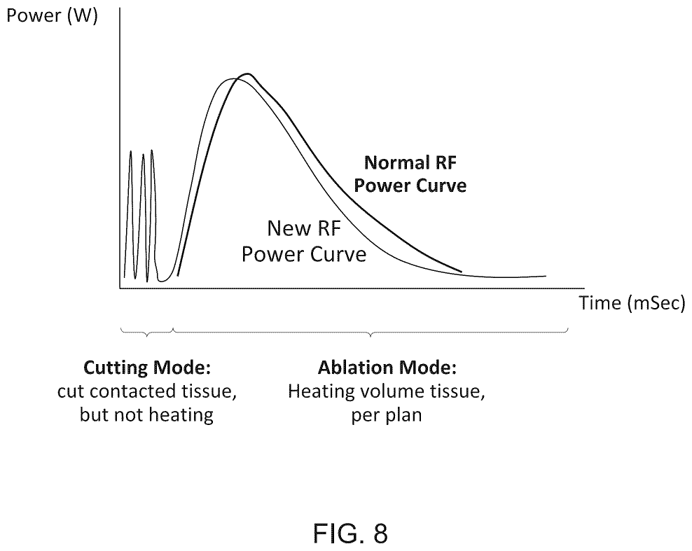

In some embodiments, the cutting or insertion mode is configured to help the ablation element cut or penetrate through target tissue. The cutting or insertion mode can be added as a pre-ablation step to ease deployment of the ablation element (e.g., an introducer or electrode ablation needles) within the target tissue. For example, as shown in FIG. 8, the cutting or insertion mode can provide an oscillation of power to the ablation element. In other examples described herein, the power in cutting or insertion mode is not oscillated. The RF energy of the cutting or insertion mode can be configured to allow the ablation element to cut or soften tissue contacted by the ablation element. When the setting of the RF generator is on “cut,” in some non-limiting examples the ablation element may maintain a substantially constant surface temperature and the voltage and/or current and/or power may be oscillated. This can cause a rapid increase in temperature in the tissue in contact with the ablation element, which can lead to tissue vaporization and cutting. The oscillating power or voltage from the RF generator can heat (and in some embodiments, vaporize) the tissue in contact with the ablation element by inducing intracellular oscillation of ionized molecules. When in “cut” mode, the RF generator can be configured to supply sufficient energy to cut the target tissue, but not so much energy as to damage to the tip of ablation element, generate excess carbonization (i.e., create charcoal), and/or cause impedance runaway.

In some embodiments, the system for penetrating target tissue is further configured to provide mechanical vibration or cutting force to the ablation element during the cutting or insertion mode. The extra mechanical vibration or cutting force, in addition to the “cut” mode RF energy, can further facilitate the insertion of the ablation element into the target tissue. In some embodiments, the system can detect a force on the ablation element when it is in contact with (e.g., being pushed into) the target tissue; and in some embodiments, the RF generator's delivery of RF energy to the ablation element is controlled based on detection of the force. In such an embodiment, for example, RF energy could be delivered to the ablation element according to the cutting or insertion mode when the ablation element is applying a forward force (e.g., being pushed into) the target tissue; then when the pressure is temporarily removed, the delivery of RF energy to the ablation element would temporarily stop. Such a force-or pressure-based feedback mechanism can reduce the heating of the target tissue while the RF generator is in cutting or insertion mode.

Embodiments of the present disclosure of the “cut” mode have been verified on ablation surrogates in multiple bench tests. It has been observed that the “hot” tip (e.g., the ablation element receiving RF energy from the RF generator in “cut” mode) can soften the target tissue and help insertion of the ablation element during uterine ablation procedures. In short, the addition of a cutting or insertion mode assists an ablation element (e.g., an introducer and/or a plurality of electrode ablation needles) to penetrate layers of target tissue while they are being deployed before ablation.

In some embodiments, the ablation/coagulation mode is configured to ablate and/or coagulate the target tissue. The ablation mode can be similar to the mode disclosed in FIG. 7D. In some embodiments, when set to “ablation” mode, the RF generator provides for an increase in energy (e.g., an increase in voltage) until the target tissue reaches a target temperature, after which time the energy (e.g., the voltage) provided from the RF generator to the ablation elements decreases. In a given target temperature setting (degree) with an RF generator, the voltage may be increased initially and start to gradually decrease after reach the target temperature. Compared to the “cut” mode, the result of the “ablation” mode may be a slower but deeper rise in tissue temperature and collagen denaturation.

FIG. 8 shows a graph of power over time of each of a cutting or insertion mode and an ablation or coagulation mode in treating target tissue, in accordance with some embodiments. In some embodiments, while the RF generator can be configured to be controlled to provide limits on temperature during each the “cut” and “ablation” modes, the limit on temperature during the cutting or insertion mode can be lower than the limit on temperature during the ablation or coagulation mode. The power curve shown in FIG. 8 emphasizes that in some embodiments, when the RF generator is in cut mode, the ablation element-receiving oscillating power from the RF generator in this example-cuts contacted tissue but does not substantially heat the tissue surrounding the ablation element. Meanwhile, when the RF generator is in ablation mode, the ablation elements heat (e.g., ablate) the volume of tissue surrounding the ablation elements.

In some embodiments, the system can further include an ultrasonic imaging device. The ultrasonic imaging device can be configured to provide for visualization of the ablation instrument as the ablation instrument penetrates the target tissue. The ultrasonic imaging device can be similar to the imaging transducer disclosed herein above (for example, the imaging transducer 107 shown in FIG. 2). As disclosed elsewhere above, the ultrasonic imaging device can be coupled to the ablation instrument. One example of an ultrasonic imaging device being coupled to an ablation instrument is shown in FIG. 2.

In some embodiments, the system can also include a controller. The controller can be designed to control the delivery of energy to the ablation element. The controller can be similar to the system controller 612 shown in FIG. 6. As disclosed above and shown in FIG. 6, the instrument 300 may be connected to and in communication with the controller 612 to provide one or more of a control signal, a feedback signal, a position signal, or a status signal. In some embodiments, the controller can be configured to monitor a temperature measured by the ablation instrument at an interface with the target tissue. In some embodiments, the controller can also be configured to maintain the temperature of about, at least about, or no more than about 80° C., 85° C., 90° C., 95° C., 100° C., 105° C., 110° C., 115° C., or more or less, and ranges including any two of the foregoing values, as the ablation element penetrates into the target tissue. In some embodiments, the controller 612 can be configured to provide an alert when the temperature at the interface is within a certain temperature range. The alert can provide an indication to begin penetration by the ablation instrument into the target tissue. In some embodiments, the temperature range can be, for example, between 80° C. and 115° C., between 80° C. and 110° C., between 80° C. and 105° C., between 80° C. and 100° C., or between 85° C. and 105° C. The alert can be anything that a user (e.g., a physician) can notice, such as a noise, an illumination or color change of a light bulb, or a tactile vibration.

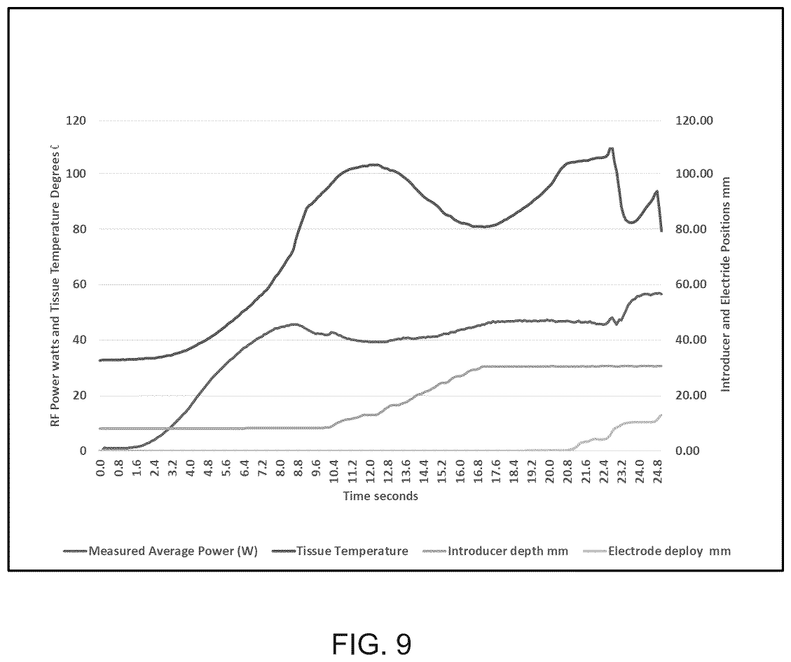

In some embodiments, the controller 612 can maintain the temperature measured by the ablation instrument by modulating power delivered to the ablation instrument. In some embodiments, the controller 612 can modulate the power delivered to the ablation instrument as the ablation instrument penetrates into the target tissue to provide an oscillation in the temperature at the interface with the target tissue. In some embodiments, the controller 612 can be configured to control the delivery of energy to the ablation instrument to ablate the target tissue after the ablation instrument has penetrated into the target tissue. In some embodiments, controlling the delivery of energy to the ablation instrument to ablate the target tissue can include maintaining a substantially constant temperature at the target tissue to ablate the tissue. Since the target tissue can hold heat, over the course of the tissue ablation, the energy delivered to the ablation element may be decreased in order to maintain a substantially constant temperature at the target tissue. One example of this can be seen in FIG. 9. After roughly 63 seconds in the example shown in FIG. 9, the power delivered to the ablation element is decreased while the temperate at the target tissue is maintained substantially constant.

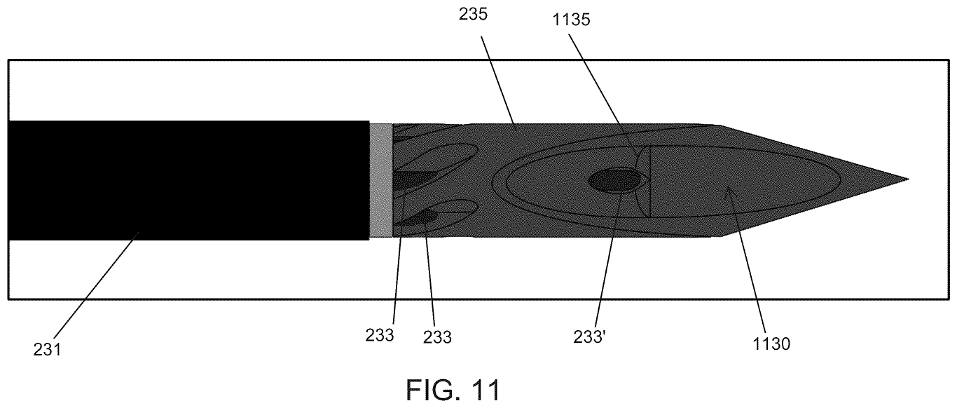

In embodiments in which the temperature of tissue is monitored, the ablation instrument can comprise one or more thermocouples. One example of a position where a thermocouple can be provided is shown in FIG. 11. FIG. 11 shows a schematic side-sectional view of the distal tip of an introducer 235. The introducer 235 is shown in an at least partially extended position. The introducer 235 is provided at the distal end of a shaft 231 of an ablation instrument. FIG. 11 further shows a plurality of electrode ablation needles or tines 233 shown in their retracted positions within the introducer 235. The plurality of electrode ablation needles or tines 233 can include a central electrode ablation needle or tine 233′. Whereas the remaining electrode ablation needles or tines 233 can be deployed at least partially radially outwardly from the introducer 235 (as shown in FIG. 2), the central electrode ablation needle or tine 233′ can be deployed substantially parallel to and in line with the introducer 235.

In some embodiments, the temperature measured by the ablation instrument as the ablation instrument penetrates into the target tissue can be measured by a thermocouple positioned on one of the electrode ablation needles or tines 233 when the plurality of electrode ablation needles or tines 233 is retracted within the introducer 235. In some embodiments, the thermocouple is positioned on the central electrode ablation needle or tine 233′.

In some embodiments, while in its retracted position within the introducer 235, the central electrode ablation needle or tine 233′ can be in contact with the tissue. In some embodiments, as shown in FIG. 11, the central tine 233′ can protrude slightly beyond a central lip 1135 of the introducer 235 into a valley surface 1130 of the introducer 235. To ensure that the central tine 233′ can be deployed from within the introducer 235, a central opening can be provided in the introducer 235. The central tine 233′ can be deployed from the introducer 235 through the central opening, and the central tine 233′can be retracted through the central opening back into the introducer 235. As shown in FIG. 11, a distal portion of the central opening can be uncovered (e.g., exposed to contact tissue), while a proximal portion of the central opening can be covered (e.g., not exposed to contact tissue). The surface of the introducer 235 that is uncovered (e.g., exposed to contact tissue) is the valley surface 1130 of the introducer 235. The distal edge of the portion of the introducer that covers the covered portion of the central opening is the central lip 1135 of the introducer 235. In some embodiments, the central tine 233′ can protrude slightly beyond a central lip 1135 of the introducer 235, such that the distalmost tip of the central tine 233′ is substantially in line with the central lip 1135 of the introducer 235 or is just slightly uncovered. This distalmost tip of the central tine 233′ can be in intimate contact with the surrounding tissue, and the thermocouple can be positioned on the central electrode ablation needle or tine 233′.

A system for penetrating target tissue like the one described above can be used in methods of fibroid ablation (e.g., uterine fibroid ablation). Some embodiments of fibroid ablation treatment are depicted in FIGS. 8, 9, 10, and 12. First, an ablation element is deployed into the fibroid. In some embodiments of a method of fibroid ablation, an ablation element is delivered into contact with a fibroid (e.g., a uterine fibroid). While the ablation element is in contact with the fibroid, RF energy can be delivered to the ablation element according to a cutting or insertion mode. In the cutting or insertion mode, according to some examples, a power can be oscillated to assist the ablation element in penetrating into the fibroid. One embodiment of this power delivery over time is shown in the “cutting mode” section of FIG. 8. In some embodiments, the delivery of the RF energy according to the cutting or insertion mode is controlled to a maximum output not to exceed, for example, about, at least about, or no more than about 50 W, 55 W, 60 W, 65 W, 70 W, 75 W, 80 W. In some embodiments, the delivery of the RF energy according to the cutting or insertion mode is controlled to not exceed, for example, 10 seconds, 15 seconds, 20 seconds, 25 seconds, 30 seconds, or 40 seconds. In some embodiments, RF energy is delivered to the ablation element in the cutting or insertion mode so that a substantially constant temperature is maintained at the surface of the ablation element. In some embodiments, the substantially constant temperature of the ablation element can be, for example, between 80° C. and 115° C. In some embodiments, the substantially constant temperature at which the surface of the ablation element is maintained during the cutting or insertion mode is lower than a target temperature, where the target temperature is the temperature of tissue in contact with the ablation element during ablation. The delivery of RF energy according to the cutting or insertion mode during deployment of the ablation element into the fibroid can soften the tissue of the fibroid. This softening of the fibroid tissue can reduce the force required for the ablation element to penetrate the fibroid. And reducing the required penetration force allows the ablation element to penetrate the fibroid without, for example, substantially deforming the fibroid upon penetration. Assisted by the cutting or insertion mode, the ablation element can be deployed into the fibroid.

FIG. 9 shows one embodiment of a method of using a system for penetrating target tissue according to another example. According to the embodiment depicted in FIG. 9, to begin the cutting or insertion, power can be increased to increase the monitored temperature of the tissue in contact with the ablation instrument. After roughly 10 seconds, once the monitored temperature reaches roughly 90° C. (or about 80° C., in another example), the introducer starts to be deployed to begin penetration into the target tissue. For example, the introducer is deployed over roughly 8 seconds to a position of roughly 33 mm. For roughly 4 seconds thereafter, the monitored temperature of the tissue increases from roughly 80° C. to roughly 100° C. At the time of roughly 21 seconds, the electrode ablation needles or tines start to be deployed from the introducer. In this example, the electrode ablation needles or tines advance roughly 12 mm in about 5 seconds. As illustrated, the temperature may oscillate based on the modulation of power and is not maintained constant during the cutting or insertion mode, and is preferably maintained in one example in a range between about 80° C. and about 115° C. during penetration of the ablation element(s).

It will be understood that FIG. 9 is an illustrative example. For example, while the cutting or insertion power of FIG. 9 is roughly 40-50 W, more or less power can be applied. In addition, the process of cutting or inserting can take more or less than 25 seconds. For example, the cutting or insertion may take more time if less power is used, while the cutting or insertion may take less time if more power is used or if a procedure calls for incomplete deployment of the electrode ablation needles.

Second, once the ablation element(s) are inserted into the target tissue, the ablation element(s) ablate the fibroid. After the ablation element penetrates into the fibroid and is deployed therein, RF energy can be delivered to the ablation element according to a coagulation or ablation mode. In the coagulation or ablation mode, a voltage or power can be applied to the ablation element to increase and then decrease after tissue in contact with the ablation element reaches a target temperature. In some embodiments, the target temperature can be, for example, between 80° C. and 115° C. In some embodiments, the fibroid is maintained at the target temperature throughout the ablation or coagulation mode. One embodiment of this power delivery over time is shown in the “ablation mode” section of FIG. 8. Once ablation of the fibroid is complete, the system and devices being used can, for example, be moved to another fibroid for ablation or removed from the procedure region (e.g., removed from the uterus).

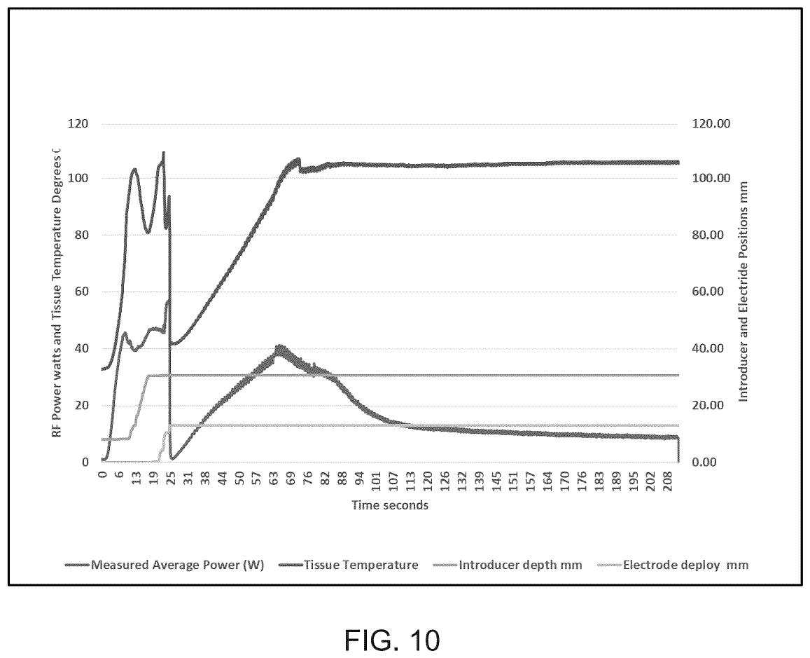

FIG. 10 shows one embodiment of a method of using a system for ablating target tissue. The graph shown in FIG. 10 shows the same example depicted in FIG. 9, but FIG. 10 shows both the cutting or insertion of the ablation elements as well as the ablation procedure. The first roughly 25 seconds of FIG. 10 match the graph shown in FIG. 9. At approximately 25 seconds, the ablation elements have been inserted into the target tissue and ablation is shown to begin. For approximately 40 seconds, power is increased, and tissue temperature correspondingly increases. Once the tissue reaches a target ablation temperature (in FIG. 10, between 100° C. and 110° C.), the system can modulate the energy delivered to the tissue in order to keep the temperature substantially constant at the target ablation temperature. As shown in FIG. 10, the power decreases over a period of approximately 40 seconds to a power of roughly 10 W, while the monitored temperature remains substantially constant. This continues for the duration of the procedure. Once the procedure is complete, the ablation elements can be retracted.

It will be understood that FIG. 10 is an illustrative example. For example, the ablation temperature shown in FIG. 10 is between 100° C. and 110° C., but that temperature can be higher or lower according to this disclosure.

FIG. 10 depicts two regimes: the first approximately 25 seconds depict a cutting or insertion mode, and the remaining time depicts an ablation or coagulation mode. It will be understood that while ablation can immediately follow cutting or insertion, there may be a period of time (e.g., 30 or 60 or fewer or more seconds) after the cutting or insertion is complete before the ablation is commenced. This period, for example, can be used to plan or otherwise prepare for the ablation procedure.

While the “cutting” and “ablating” modes or regimes can be distinct from one another, in some embodiments, the delivery of RF energy according to the cutting or insertion mode can also preheat the uterine fibroid, which can in turn reduce the time required for the ablation procedure.

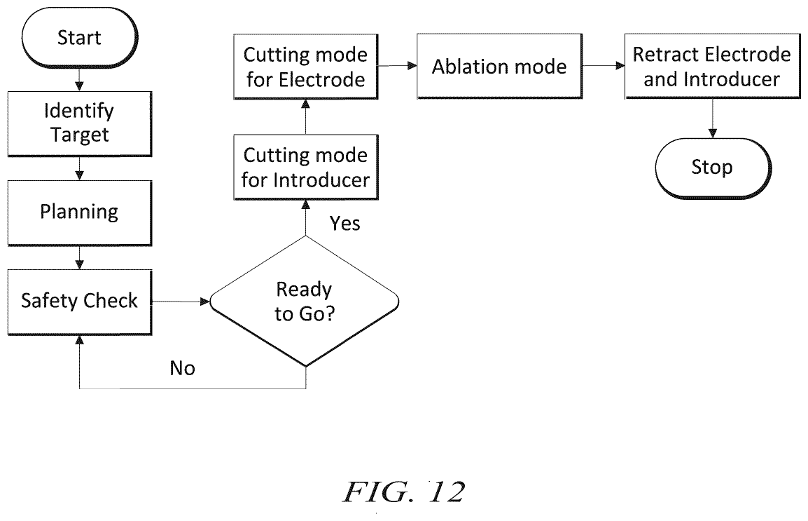

Additionally, while the “cutting” and “ablation” steps of the method are detailed above, an ablation procedure may include additional steps, outlined in the flow-chart of FIG. 12. In some embodiments, for example, before the ablation element can be deployed into a fibroid, the target tissue (e.g., fibroid) is identified. In some embodiments, the target tissue can be identified using the imaging component 100, as shown, for example, in FIGS. 1A, 3A, 3B, 4, 5A-5C, and 6. In some embodiments, after identifying the target, the ablation procedure is planned. For example, the proper equipment is prepared. In some embodiments, planning can include providing the ablation element adjacent to the fibroid. In some embodiments in which the ablation element is an ablation instrument similar to the ablation instrument 230 shown in FIG. 2, the placement of the ablation instrument adjacent to the fibroid can look like FIG. 7A. A safety check can then be performed to ensure that the target tissue can be safely penetrated and/or ablated. In embodiments in which the ablation element is similar to the ablation instrument 230 shown in FIG. 2, the next step can include deploying the introducer (235 in FIG. 7C). Then, in some embodiments, the next step can include deploying the electrode needles or tines (233 in FIG. 7D). Deploying the introducer and/or deploying the electrode needles can be done according to the “cutting or insertion mode” detailed above. Once the ablation element is adequately deployed into the fibroid, the fibroid can be ablated, as disclosed herein. Ablating the fibroid can be done according to the “ablation mode” detailed above. In some embodiments, once the fibroid ablation is complete, the electrode needles and introducer can be retracted. Then the system and devices can, for example, be moved to another fibroid for ablation or removed from the procedure region (e.g., removed from the uterus).

Embodiments of the present disclosure are applicable to the Sonata® System available from Gynesonics, Inc. of Redwood City, CA and like systems, devices, and methods described in the following co-assigned U.S. Patents and Patent Applications, which are incorporated herein by reference: U.S. Pat. Nos. 7,918,795; 9,357,977; 7,815,571; 7,874,986; 10,058,342; 8,088,072; 8,206,300; 9,861,336; 8,992,427; 11,219,483; U.S. Pat. Pub. 2020/0275975 (now U.S. Pat. No. 11,612,431); U.S. Pat. Pub. 2021/0228179 (now U.S. Pat. No. 11,583,243) and U.S. Pat. Pub. 2019/0350648.

The foregoing description and examples has been set forth merely to illustrate the disclosure and are not intended as being limiting. Each of the disclosed aspects and embodiments of the present disclosure may be considered individually or in combination with other aspects, embodiments, and variations of the disclosure. In addition, unless otherwise specified, none of the steps of the methods of the present disclosure are confined to any particular order of performance. Modifications of the disclosed embodiments incorporating the spirit and substance of the disclosure may occur to persons skilled in the art and such modifications are within the scope of the present disclosure.

Terms of orientation used herein, such as “top,” “bottom,” “horizontal,” “vertical,” “longitudinal,” “lateral,” and “end” are used in the context of the illustrated embodiment. However, the present disclosure should not be limited to the illustrated orientation. Indeed, other orientations are possible and are within the scope of this disclosure. Terms relating to circular shapes as used herein, such as diameter or radius, should be understood not to require perfect circular structures, but rather should be applied to any suitable structure with a cross-sectional region that can be measured from side-to-side. Terms relating to shapes generally, such as “circular” or “cylindrical” or “semi-circular” or “semi-cylindrical” or any related or similar terms, are not required to conform strictly to the mathematical definitions of circles or cylinders or other structures, but can encompass structures that are reasonably close approximations.

Conditional language used herein, such as, among others, “can,” “might,” “may,” “e.g.,” and the like, unless specifically stated otherwise, or otherwise understood within the context as used, is generally intended to convey that some embodiments include, while other embodiments do not include, certain features, elements, and/or states. Thus, such conditional language is not generally intended to imply that features, elements, blocks, and/or states are in any way required for one or more embodiments or that one or more embodiments necessarily include logic for deciding, with or without author input or prompting, whether these features, elements and/or states are included or are to be performed in any particular embodiment.

Conjunctive language, such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

The terms “approximately,” “about,” and “substantially” as used herein represent an amount close to the stated amount that still performs a desired function or achieves a desired result. For example, in some embodiments, as the context may dictate, the terms “approximately”, “about”, and “substantially” may refer to an amount that is within less than or equal to 10% of the stated amount. The term “generally” as used herein represents a value, amount, or characteristic that predominantly includes or tends toward a particular value, amount, or characteristic. As an example, in certain embodiments, as the context may dictate, the term “generally parallel” can refer to something that departs from exactly parallel by less than or equal to 20 degrees.

Where term “about” is utilized before a range of two numerical values, this is intended to include a range between about the first value and about the second value, as well as a range from the first value specified to the second value specified.

Unless otherwise explicitly stated, articles such as “a” or “an” should generally be interpreted to include one or more described items. Accordingly, phrases such as “a device configured to” are intended to include one or more recited devices. Such one or more recited devices can be collectively configured to carry out the stated recitations. For example, “a processor configured to carry out recitations A, B, and C” can include a first processor configured to carry out recitation A working in conjunction with a second processor configured to carry out recitations B and C.

The terms “comprising,” “including,” “having,” and the like are synonymous and are used inclusively, in an open-ended fashion, and do not exclude additional elements, features, acts, operations, and so forth. Likewise, the terms “some,” “certain,” and the like are synonymous and are used in an open-ended fashion. Also, the term “or” is used in its inclusive sense (and not in its exclusive sense) so that when used, for example, to connect a list of elements, the term “or” means one, some, or all of the elements in the list.

Overall, the language of the claims is to be interpreted broadly based on the language employed in the claims. The language of the claims is not to be limited to the non-exclusive embodiments and examples that are illustrated and described in this disclosure, or that are discussed during the prosecution of the application.

Although systems, devices, and methods for endovascular implants and accurate placement thereof have been disclosed in the context of certain embodiments and examples, this disclosure extends beyond the specifically disclosed embodiments to other alternative embodiments and/or uses of the embodiments and certain modifications and equivalents thereof. Various features and aspects of the disclosed embodiments can be combined with or substituted for one another in order to form varying modes of systems, devices and methods for endovascular implants and accurate placement thereof. The scope of this disclosure should not be limited by the particular disclosed embodiments described herein.

Certain features that are described in this disclosure in the context of separate implementations can be implemented in combination in a single implementation. Conversely, various features that are described in the context of a single implementation can be implemented in multiple implementations separately or in any suitable subcombination. Although features may be described herein as acting in certain combinations, one or more features from a claimed combination can, in some cases, be excised from the combination, and the combination may be claimed as any subcombination or variation of any subcombination.

While the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various embodiments described and the appended claims. Further, the disclosure herein of any particular feature, aspect, method, property, characteristic, quality, attribute, element, or the like in connection with an embodiment can be used in all other embodiments set forth herein. Any methods disclosed herein need not be performed in the order recited. Depending on the embodiment, one or more acts, events, or functions of any of the algorithms, methods, or processes described herein can be performed in a different sequence, can be added, merged, or left out altogether (e.g., not all described acts or events are necessary for the practice of the algorithm). In some embodiments, acts or events can be performed concurrently, e.g., through multi-threaded processing, interrupt processing, or multiple processors or processor cores or on other parallel architectures, rather than sequentially. Further, no element, feature, block, or step, or group of elements, features, blocks, or steps, are necessary or indispensable to each embodiment. Additionally, all possible combinations, subcombinations, and rearrangements of systems, methods, features, elements, modules, blocks, and so forth are within the scope of this disclosure. The use of sequential, or time-ordered language, such as “then,” “next,” “after,” “subsequently,” and the like, unless specifically stated otherwise, or otherwise understood within the context as used, is generally intended to facilitate the flow of the text and is not intended to limit the sequence of operations performed. Thus, some embodiments may be performed using the sequence of operations described herein, while other embodiments may be performed following a different sequence of operations.

Moreover, while operations may be depicted in the drawings or described in the specification in a particular order, such operations need not be performed in the particular order shown or in sequential order, and all operations need not be performed, to achieve the desirable results. Other operations that are not depicted or described can be incorporated in the example methods and processes. For example, one or more additional operations can be performed before, after, simultaneously, or between any of the described operations. Further, the operations may be rearranged or reordered in other implementations. Also, the separation of various system components in the implementations described herein should not be understood as requiring such separation in all implementations, and it should be understood that the described components and systems can generally be integrated together in a single product or packaged into multiple products. Additionally, other implementations are within the scope of this disclosure.

Some embodiments have been described in connection with the accompanying figures. Certain figures are drawn and/or shown to scale, but such scale should not be limiting, since dimensions and proportions other than what are shown are contemplated and are within the scope of the embodiments disclosed herein. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, any methods described herein may be practiced using any device suitable for performing the recited steps.

The methods disclosed herein may include certain actions taken by a practitioner; however, the methods can also include any third-party instruction of those actions, either expressly or by implication. For example, actions such as “positioning an electrode” include “instructing positioning of an electrode.”

In summary, various embodiments and examples of endovascular implants and devices and methods for accurate placement have been disclosed. Although the systems, devices and methods for endovascular implants and accurate placement thereof have been disclosed in the context of those embodiments and examples, this disclosure extends beyond the specifically disclosed embodiments to other alternative embodiments and/or other uses of the embodiments, as well as to certain modifications and equivalents thereof. This disclosure expressly contemplates that various features and aspects of the disclosed embodiments can be combined with, or substituted for, one another. Thus, the scope of this disclosure should not be limited by the particular disclosed embodiments described herein, but should be determined only by a fair reading of the claims that follow.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ═15%, etc.). For example, “about 1 V” includes “1 V.” Phrases preceded by a term such as “substantially” include the recited phrase and should be interpreted based on the circumstances (e.g., as much as reasonably possible under the circumstances). For example, “substantially perpendicular” includes “perpendicular.” Unless stated otherwise, all measurements are at standard conditions including temperature and pressure.

What is claimed is:

1. A system for penetrating target tissue, the system comprising:
an ablation instrument; and
a controller configured to control the delivery of energy to the ablation instrument, wherein the controller is configured to monitor a temperature measured by the ablation instrument at an interface with the target tissue and maintain the temperature between about 80° C. and about 115° C. as the ablation instrument penetrates into the target tissue.

2. The system of claim 1, wherein the ablation instrument comprises an introducer and a plurality of electrode ablation needles extendible from a retracted position within the introducer.

3. The system of claim 2, wherein the temperature measured by the ablation instrument is measured by a thermocouple positioned on one of the electrode ablation needles when the plurality of electrode ablation needles is retracted within the introducer.

4. The system of claim 3, wherein the plurality of electrode ablation needles comprises a central electrode extendible from a retracted position within the introducer, and wherein the thermocouple is positioned on the central electrode.

1. ystem of claim 1, wherein the controller is configured to modulate power delivered to the ablation instrument to maintain the temperature between about 80° C. and about 115° C. as the ablation instrument penetrates into the target tissue.

6. The system of claim 1, wherein the controller is configured to modulate power delivered to the ablation instrument as the ablation instrument penetrates into the target tissue to provide an oscillation in the temperature at the interface with the target tissue.

7. The system of claim 1, wherein the controller is configured to provide an alert when the temperature at the interface is between about 80° C. and about 115° C. to provide an indication to begin penetration by the ablation instrument into the target tissue.

8. The system of claim 1, wherein the system further comprises an ultrasonic imaging device configured to provide for visualization of the ablation instrument as the ablation instrument penetrates the target tissue.

9. The system of claim 8, wherein the ultrasonic imaging device is coupled to the ablation instrument.

10. The system of claim 1, wherein the controller is configured to control the delivery of energy to the ablation instrument to ablate the target tissue after the ablation instrument has penetrated into the target tissue.

11. The system of claim 10, wherein the controller is configured to control the delivery of energy to the ablation instrument to maintain a substantially constant temperature at the target tissue to ablate the target tissue.

12. The system of claim 1, further comprising a radiofrequency generator configured to deliver energy to the ablation instrument while the ablation instrument penetrates into the target tissue.

13. A method of penetrating target tissue, the method comprising:
delivering an ablation instrument into contact with a target tissue;
delivering energy to the ablation instrument to cause a temperature measured by the ablation instrument at an interface with the target tissue to rise to between about 80° C. and about 115° C.; and
advancing the ablation instrument to penetrate into the target tissue while modulating the power to maintain the temperature at the interface with the target tissue between about 80° C. and about 115° C.

14. The method of claim 13, wherein the ablation instrument penetrates into the target tissue while the temperature maintained at the interface with the target tissue oscillates.

15. The method of claim 13, further comprising ablating the target tissue by delivering energy to the ablation instrument to maintain the temperature at the interface with the target tissue at a substantially constant temperature.

16. The method of claim 13, wherein the ablation instrument comprises an introducer and a plurality of electrode ablation needles extendible from a retracted position within the introducer, wherein the plurality of electrode ablation needles is at least partially extended from the introducer while maintaining the temperature at the interface with the target tissue between about 80° C. and about 115° C.

17. A system for penetrating target tissue, the system comprising:
an ablation element configured to penetrate the target tissue; and
a radiofrequency generator configured to deliver energy to the ablation element, wherein the radiofrequency generator comprises:
a cutting or insertion mode configured to cut through target tissue, wherein the cutting or insertion mode provides for a power oscillation configured to cut tissue contacted by the ablation element; and
an ablation or coagulation mode configured to ablate and/or coagulate the target tissue, wherein the ablation or coagulation mode provides for an increase in power and then a decrease in power after the target tissue reaches a target temperature.

18. The system of claim 17, wherein the cutting or insertion mode is configured to maintain a substantially constant surface temperature of the ablation element.

19. The system of claim 17, wherein the cutting or insertion mode is configured to cause a rapid increase in temperature in tissue in contact with the ablation element.

20. The system of claim 17, wherein the radiofrequency generator is configured to be controlled to provide a limit on temperature during the cutting or insertion mode and during the ablation or coagulation mode, wherein the limit on temperature during the cutting or insertion mode is lower than the limit on temperature during the ablation or coagulation mode.

21. The system of claim 17, further configured to provide a mechanical vibration or cutting force during the cutting or insertion mode.

22. The system of claim 17, wherein the ablation element comprises an introducer.

23. The system of claim 17, wherein the ablation element comprises a plurality of electrode ablation needles.

24. The system of claim 17, further comprising a controller configured to control the delivery of energy to the ablation element, wherein the controller is configured to monitor a temperature measured by the ablation element at an interface with the target tissue and maintain the temperature between about 80° C. and about 115° C. as the ablation element penetrates into the target tissue.

25. The system of claim 17, wherein the target tissue is a uterine fibroid.

26. A method of uterine fibroid ablation, comprising:
delivering an ablation element into contact with a uterine fibroid;
delivering radiofrequency energy according to a cutting or insertion mode to the ablation element while the ablation element is in contact with the uterine fibroid, wherein in the cutting or insertion mode a voltage or power is modulated based on a temperature measured at an interface between the ablation element and the uterine fibroid to assist the ablation element in penetrating into the uterine fibroid; and
after the ablation element penetrates into the uterine fibroid, delivering radiofrequency energy according to a coagulation mode, wherein in the coagulation mode a voltage or power applied to the ablation element is sufficient to ablate the uterine fibroid.

27. The method of claim 26, wherein the voltage or power is oscillated in the cutting or insertion mode.

28. The method of claim 26, wherein the voltage or power is modulated in the cutting or insertion mode to maintain a temperature at the interface between about 80° C. and about 115° C.

29. The method of claim 26, wherein the voltage or power in the coagulation mode increases and then decreases after tissue in contact with the ablation element reaches a target temperature.

30. The method of claim 26, wherein delivering radiofrequency energy according to the cutting or insertion mode preheats the uterine fibroid.

31. The method of claim 26, wherein delivering radiofrequency energy according to the cutting or insertion mode softens the uterine fibroid thereby allowing the ablation element to penetrate the uterine fibroid without substantially deforming the uterine fibroid upon penetration.

32. The method of claim 26, wherein delivering radiofrequency energy according to the cutting or insertion mode maintains a substantially constant surface temperature of the ablation element.

33. The method of claim 32, wherein the substantially constant surface temperature of the ablation element is between about 80° C. and about 115° C.

34. The method of claim 26, wherein delivering radiofrequency energy according to the cutting or insertion mode is controlled to a maximum output not to exceed 70 watts.

35. The method of claim 26, wherein delivering radiofrequency energy according to the cutting or insertion mode is controlled not to exceed 30 seconds.

36. The method of claim 26, further comprising detecting a force on the ablation element when in contact with the uterine fibroid, and wherein delivering radiofrequency energy according to the cutting or insertion mode is controlled based on detection of the force.
